(19) United States
(12) Patent Application Publication
(10) Pub. No.: US 2004/0115683 A1
(43) Pub. Date: Jun. 17, 2004

(54) Analysis of gene expression and biological function using optical imaging

(76) Inventors: June Iris Medford, Fort Collins, CO (US); Ronald Lee Parsons, Fort Collins, CO (US); Aaron Reeves, Loveland, CO (US)

Correspondence Address: Winner & Sullivan, Suite 201, 5370 Manhattan Circle, Boulder, CO, 80303, US

(21) Appl. No.: 10/470,211

(22) PCT Filed: Jan. 28, 2002
(86) PCT No.: PCT/US02/02414

(51) Int. Cl. C12Q 1/68; G06F 19/00; G01N 33/48; G01N 33/50

(52) U.S. Cl. 435/6; 702/20

(57) ABSTRACT

The present invention provides methods, composition, software and apparatus for in vivo analysis of gene expression and biological function in plants and animals. The invention also provides materials and methods for rapid analysis of data, allowing the methods and compositions of the invention to be used in a high throughput manner. The materials include an optical coherence microscope, the use and identification of detectable OCM substances, and machine readable code that converts OCM signal to binary data and then to a visual image, by which the expression of genes in vivo can be visualized. The present method results in the creation of genomic-scale, computer screenable databases of gene expression that include spatial, temporal, and quantitative data about gene expression in vivo.

### CROSS-REFERENCE TO RELATED APPLICATIONS

This application claims priority from U.S. Provisional Applications 60/264,641 and 60/264,450, both filed Jan. 26, 2001. These provisional applications are Incorporated by reference in their entirety herein.

### STATEMENT REGARDING GOVERNMENT FUNDING

Not applicable.

### FIELD OF THE INVENTION

The present invention relates to the field of genomics and biotechnology, and materials and methods useful for analyzing gene expression in a spatial, temporal and quantitative manner. The method of the present invention is particularly well-suited to in vivo detection of gene expression.

### BACKGROUND OF THE INVENTION

How genes are expressed is a fundamental question in biology. An unmet challenge is to examine three-dimensional gene expression in intact organisms without disruption. Tissue destruction and extraction are currently the only methods by which genomic-scale expression studies can be done (e.g., microarrays, RT-PCR, gel blots), but these methods introduce artifacts from the extractions and other manipulations and spatial information about the gene expression is lost.

Many examples where the ability to analyze gene expression in living intact organisms would make an impact can be described. Two illustrations: A fundamental process in developmental biology is organ formation. In plants, a leaf forms as a mound of cells on the side of a shoot meristem (Steeves, T. A., and Sussex, I. M. (1989) Cambridge University Press). The mound flattens, develops three distinct axes (dorsoventral, lateral, proxo-distal) and the leaf blade and leaf petiole differentiate. Some of the genes involved in these events have been characterized (Clark, S. E., et al. (1996) Development 122, 1567-75; Fletcher, J. C., et al. (1999) Science 283, 1911-4; Lynn, K., et al. (1999) Development 126, 469-81; Siegfried, K. R., et al. (1999) Development 126, 4117-28; Timmermans, M. C. P., et al. (1998) Development 125, 2813-2823; Trotochaud, A. E., et al. (1999) Plant Cell 11, 393-406; and Trotochaud, A. E., et al. (2000) Science 289, 613-7). However, our knowledge for how these genes function in organ formation must be inferred from discontinuous “snap-shots” analyzed from different plants. Such “snap-shot” understanding is also true for our knowledge of animal and human processes. A second example is the invasion of plants and animals by pathogens. In the case of plant pathogens, the pathogen is well known to induce a response throughout the entire organism. For example, two hours after wounding a leaf, the promoter of a gene involved in the production of a wound signal, salicylic acid, is induced in the leaf blade (Kubigsteltig, I., et al. (1999) Planta 208, 463-71). Within 24 hours, the gene has been induced throughout the entire plant. Accordingly, a signal has been transduced through the leaf blade and petiole to the stem. In the stem, or prior to this, the signal must be transmitted three dimensionally. But how is this done? How does a signal move three-dimensionally in a whole organism? Does the signal transduction machinery transmit a signal at all angles or is does it follow x-y-z vectors? Does the signal move through a pre-established pathway, vascular tissue, or non-discriminately through all cells or perhaps some combination to allow a signal transduction to be fine-tuned within the intact organism? Technology to dynamically follow gene expression, particularly in a three dimensional manner, would provide great insight into many biological processes.

In addition to gene expression, mutant analysis has played a key rule in the understanding of genomic information and how genomic information functions. Detailed observations are key for mutant identification and analysis of biological response. Currently, observations must be performed individually or using semi-automated process e.g., with CCD cameras that only record exterior changes (Boyes, D. C., et al. (2001) Plant Cell 13, 1499-510.). However, many biological responses involve subtle changes in sub-surface cells, tissues or morphology that would not be detected with the CCD cameras. Genomic studies would be significantly advanced by instrumentation that allows gene expression and biological features to be studied in vivo while maintaining spatial integrity of the living organism and allow both of these aspects to be done in a high throughput manner. Another difficulty with genomic-scale studies is analysis of the enormous amounts of data generated and how to accurately identify important aspects of the information that data contain.

Limited technologies for in vivo imaging of gene expression exist. However, these technologies are limited by depth penetration (confocal) or resolution (MRI and PET). The present fills an important “niche” for in vivo imaging of gene expression in that it allows relatively inexpensive high resolution imaging in intact organisms.

To analyze gene expression in vivo, products of “reporter genes” are typically followed. One of the most widely used reporter gene products is green fluorescent protein (GFP) (and its variants) visualized with a confocal microscope (Billinton, N., and Knight, A. W. (2001) Anal Biochem 291, 175-97;, Hanson, M. R., and Kohler, R. H. (2001) J. Exp. Botany 52, 529-539; Haseloff, J. (1999). Methods in Cell Biology 58, 139-151; and Haseloff, J., et al. (1999) In Methods in Molecular Biology: Protocols in Confocal Microscopy, S. Paddock, ed. (Totowa, N.J.: Humana Press)). GFP visualization has been, and will continue to be, very powerful for studies with isolated cells and small transparent tissues (e.g., Arabidopsis roots, developing Zebra fish. In intact organisms, GFP use is limited to relatively shallow depths of 60-80 μm (Haseloff, J. (1999). supra; and Haseloff, J., et al. (1999). supra). Further, for GFP visualization in plants, samples are submerged in water for examination with a water immersion lens (Billinton, N., and Knight, A. W. (2001) supra; Hanson, M. R., and Kohler, R. H. (2001) supra; Haseloff, J. (1999). supra; and Haseloff, J., et al. (1999) supra). Plants viewed in this manner are probably still alive, hence the analysis is “in vivo”, but the environment is far from normal. Also, GFP imaging appears to involve the production of free radicals, which are potentially damaging to the plant (Haseloff, J. (1999) supra; and Haseloff, J., et al. (1999) supra). There is one report of GFP toxicity in living cells (Liu, H. S., et al. (1999). Biochem Biophys Res Commun 260, 712-7). For developmental regions such as the shoot apex, confocal imaging requires removal of overlying leaves (Haseloff, J. (1999) supra; Haseloff, J., et al. (1999) supra; Laufs, P., et al. (1998) The Plant Cell 10, 1375-1389; and Running, M. P., et al. (1995) Methods in Cel Biology 49, 217-229.). Also, because confocal microscopy (including multiphoton microscopy) and GFP imaging typically requires a fluorescence light source, endogenous auto-fluorescence from plant pigments and cell walls produce significant interference(Billinton, N., and Knight, A. W. (2001) Anal Biochem 291, 175-97).

Another in vivo imaging technology is PET (positron emission tomography) (Phelps, M. E. (2000). Proc Natl Acad Sci USA 97, 9226-33). The technique is powerful and has been used to produce images of living organisms. PET has been used to image gene expression in vivo using positron emitting probes (Gambhir, S. S., et al. (1999) Proc Natl Acad Sci USA96, 2333-8; Gambhir, S. S., et al. (2000) Proc Natl Acad Sci USA97, 2785-90; and Herschman, H. R., et al. (2000). J Neurosci Res 59, 699-705). However, three factors make PET (and μPET) less than practical for routine imaging of gene expression. First, the resolution is in centimeters, significantly preventing detailed analysis. Second, the cost of the instrument is prohibitive (exceeds $1 million). Third, imaging gene expression requires injection of a PET reporter probe specific for the gene of interest. The PET reporter probe requires a nearby “PET radiopharmacies with electronic generators” because PET compounds have very short half-lives (2-108 minutes) (Phelps, M. E. (2000) Proc Natl Acad Sci USA 97, 9226-33.). PET imaging of gene expression in mice and rats in vivo, required injection of the PET reporter probe into a tail vein with delivery to the area of interest (e.g., the liver) accomplished with the animal's circulatory system. For plants, it is not clear how PET reporter probes could be delivered.

Other types of gene imaging technologies include luciferase and MRI. Luciferase expression can be detected with sensitive CCD cameras and has been useful in studies involving the entire plant (e.g., circadian rhythms) and intact mouse brains, but lacks the resolution needed for analysis of gene expression in vivo (Meier, C., et al. (2001) Plant J 25, 509-19; Shi, N., et al. (2000) Proc Natl Acad Sci USA 97, 14709-14714; and Thain, S. C., et al. (2000) Curr Biol 10, 951-6). A very limited type of in vivo gene expression was recently reported with MRI (Contag, C. H., et al. (2000) NeoReviews 1, e225-e232; Louie, A. Y., et al. (2000) Nat Biotechnol 18, 321-5; and Weissleder, R., et al. (2000) Nat Med 6, 351-5). However, it does not seem likely that this method will be generally applicable for several reasons. First, the resolution is in hundreds of microns (plant and animal cells are typically from 2 to 10 microns). Second, the “gene reporter” images from a test case were far from convincing, raising doubts about sensitivity. Third, it involved a complex biochemistry and release of a “caged”, potentially toxic metal. Fourth, cost for the MRI instrument typically exceeds $1 million, significantly limiting its availability.

Optical coherence microscopy (OCM) (also called optical coherence tomography (OCT)) allows non-destructive, non-invasive, repetitive and quantitative, in vivo visualization of subsurface cells, tissues and organs in intact organisms (Boppart, S., et al. (1997) Developmental Biology 177, 54-63; Boppart, S. A., et al. (1998) Nat Med 4, 861-5; and Hettinger, J. W., et al. (2000) Plant Physiology 123, 3-15.) This method has been applied to ophthalmology, numerous medically related studies and developmental biology (Boppart, S., et al. (1997). supra; Boppart, S., et al. (1997) Proc.Natl.Acad.Sci.USA 94, 4256-4261; Fujimoto, J. G., et al. (2000) Neoplasia 2, 9-25; and Hettinger, J. W., et al. (2000) Plant Physiology 123, 3-15) OCM has also been reported as a method for studying biological function by visualizing changes in subsurface structures in biological samples (e.g. Medford, J. I. et al. (2000) PCT Publication Number WO 00/45153 and Bopart, S., et al. supra). However, the study of biological function using as described in Medford, et al. (2000) does not address the need to study expression of a specific gene and does not include high throughput means to examine the data.

### SUMMARY OF THE INVENTION

The optical coherence microscopy method of this invention has numerous advantages over current technologies for studying gene expression in numerous types of biological samples. It can image organisms (plants, animals or humans) non-destructively, for example a plant growing in soil. It currently affords a 5-10 fold greater penetration depth than confocal microscopy. OCM/OCT can penetrate dense, opaque tissues like those found in the plants, humans and animals. The method requires no tissue preparation, staining, extraction or biopsy. For example, plants are imaged growing in soil and the same plant or animal can be imaged repeatedly without damage. Animals are imaged in their natural environment, and the use of fiber optical probes has allowed the examination of internal organs and tissues (e.g., heart, intestines) (Fujimoto et al.)[Tearney, 1997 #571; Swanson, 1993 #917; Pitris, 2001 #915; Herrmann, 1998 #764; Fujimoto, 2000 #831; Fujimoto, 1998 #763; Drexler, 2001 #941; D'Amico, 2000 #953; Boppart, 2000 #954; Boppart, 1999 #768; Boppart, 1998 #785; Boppart, 1998 #766; Boppart, 1998 #762; Boppart, 1997 #590; Boppart, 1997 #589]. Such OCM/OCT instrument improvements and adaptations use would be readily applied to the method of imaging gene expression described herein. No apparent damage to the plant is found, even after 9 days of continuous imaging (Hettinger, J. W. (2001). Masters Thesis. Fort Collins: Colorado State University, pp. 216.; Hettinger, J. W., et al. (2000) Plant Physiology 123, 3-15.). Likewise there is no apparent damage to animal and human tissues. The imaging technology has been approved and used by many for in vivo imaging of human eyes (Humphrey Instruments, Inc.) Imaged organisms continue to develop normally. It is rapid, capable of collecting an image in 1 minute and the data provided produces true three-dimensional images (WO 00/45153). Finally, OCM is relatively inexpensive (approximate retail cost of parts for an instrument is $50,000).

This invention provides methods of using OCM/OCT for the non-destructive detection, measurement and imaging of gene expression or changes in cellular structure in a quantitative, temporal and spatial manner. This invention further provides machine implemented methods that allow OCM data to be analyzed quickly and easily, allow gene expression to be clearly visualized using OCM, and allow comparative analysis of gene expression between organisms. Other aspects of this invention provide systems and methods for automated high throughput analysis of gene expression and screening of large numbers of organisms.

In one embodiment of this invention, a method is provided for detecting gene expression in a sample cell, tissue, organ or organism in which the cell, tissue, organ or organism contains one or more OCM detectable substances that may be directly introduced into said organism or whose production or degradation are directed by genes in the organisms. For example OCM detectable reporter genes are genes whose products are detectable by OCM or whose products contribute to the production or degradation of one or more OCM detectable substances. The method for detecting gene expression in a cell, tissue, organ or organism comprises the steps of: acquiring OCM data for said cell, tissue, organ or organism; and analyzing the OCM data.

The OCM data may be analyzed visually, using binary data analysis methods, or a combination of both. Methods are known in the art for introducing the one or more OCM detectable reporter genes into cells, tissues or organisms.

The methods of this invention allow gene expression in organisms (plants or animals), tissues, organs, or cells (including cultured cells) to be measured quantitatively, spatially, and temporally. While the methods of this invention are particularly well suited to in vivo studies of gene expression, it will be obvious to one skilled in the art that the methods described herein are also readily applied to the investigation of samples in various states of isolation and culture as well as in vitro samples.

In another embodiment of this invention, a method for screening a plurality of biological samples is provided, comprising: acquiring OCM data for two or more reference samples; generating a mean histogram profile for the two or more reference samples; acquiring data for a biological sample of interest; generating a histogram for the organism of interest; and comparing the histogram profile to the histogram of the sample of interest. Differences between the histograms may be indicative of differences of biological function of the sample of interest relative to the reference samples. Such biological functions include gene activity or expression, response to stress or other stimuli, developmental differences, and the like.

The identification of ‘outliers’ from the reference samples is useful for detecting the presence of genetic mutations, breeding applications (development of desirable traits, elimination of undesirable traits) or distinguishing genetically modified organisms from wild type organisms.

The present invention also provides a high throughput OCM analysis system comprising: a sample holder; a CCD or other camera in optical alignment with said sample holder and connected to a computer; a translation system for moving and positioning the sample holder in x, y, and z directions; an OCM system also in optical alignment with the sample holder at a known position relative to the camera; and a computer connected to the camera, translation system and OCM system.

### BRIEF DESCRIPTION OF THE DRAWINGS

The following drawings form part of the present specification and are included to further illustrate certain aspects of the present invention, but are in no way intended to limit the scope of the claims. The invention may be better understood by reference to one or more of these drawings in combination with the detailed description of specific embodiments presented herein.

FIG. 1 illustrates a schematic of an optical coherence microscope used in this invention.

FIG. 2 is an illustration of how 3-dimensional volume elements, or voxels, acquired during OCM analysis are arranged to produce a 3-dimensional representation, or image, of the data. Voxels contain quantitative signal information as well as spatial information.

FIG. 3 is a flow chart describing steps taken for ‘normal OCM view’ and ‘gene reporter view’.

FIG. 4 is a schematic of the high throughput optical coherence microscopy system of this invention. This illustration demonstrates the system's use for high throughput analysis of plants.

FIG. 5 shows histogram data derived from the OCM data for Arabidopsis plants that are positive and negative for PHB genes. FIG. 5A shows data over the entire OCM signal range. FIG. 5B shows OCM data over a limited OCM signal range.

FIG. 6 illustrates the effects of using binary data analysis on image representations of OCM data for a plant positive for PHB and a control plant. FIG. 6A is the ‘normal OCM view’ of a 200 m cube of Arabidopsis cotyledon tissue. In the color representation, nuclei appear as red ‘dots’, dense organelles as ‘greenish-yellow’, and cytoplasm as blue. The black spaces in the volume indicate vacuoles and/or air spaces between cells. FIG. 6B is the ‘gene reporter view’ for the same tissue shown in 6A, but with the endogenous OCM signal removed, leaving only the signal from the gene reporter molecule. FIG. 6C is ‘gene reporter view’ for control tissue after removal of endogenous OCM signal. FIGS. 6D-G are optical sections of the volumes shown in 6A and 6B. 6D-E are vertical slices, 12 m thick. FIGS. 6F-G are horizontal slices, 6 um thick. In the normal OCM views (6D,F) vacuoles have no significant signal and are dark with bluish colored cytoplasm surrounding them. Signal consistent with nuclei (N) are readily apparent. FIGS. 6E and G show the gene reporter view of D and F, respectively. PHB in these plants is targeted to plastics. The gene reporter signal is not found in nuclei or vacuoles. The gene reporter signal is seen as particulate structures in the cytoplasm, consistent with localization to the chloroplasts. In G, a slight amount of signal is seen in vacuoles. This may be signal from cells beneath the surface of the slice.

FIG. 7 shows histograms of OCM data for 11-6 day old Arabidopsis shoot apices. One plant imaged (3344) produced an aberrant graph (arrow).

FIG. 8 shows visualization of the OCM histogram data for 4 of the 6 day old Arabidopsis plants of FIG. 7. This figure shows that plant 3344 has a malformed leaf primordium. Plant 3345 was imaged at a slightly different orientation than plant 3711 with no effect on overall OCM signal.

### DETAILED DESCRIPTION OF THE INVENTION

The present invention provides OCM methods for detecting, measuring and or imaging gene expression in vivo, ex vivo and in vitro in biological systems (e.g., living cells). The present invention is based at least in part on the discovery of a substance compatible with living cells or organisms which is detectable by OCM in the biological system and which can be produced as a result of gene expression, i.e. that is a direct or indirect product of gene expression, or otherwise inserted into the cell.

The OCM gene expression method of this invention enables in vivo, ex vivo and in vitro gene expression analysis including imaging and quantitative measurement in a spatial, temporal manner. Gene expression analysis using the methods of this invention can be used in a variety of research and clinical applications and numerous analytical scenarios including, but not limited to:

Comparison of gene expression between wild type organisms;

Comparing gene expression in one or more wild type (WT) organisms (non-transgenic) to expression in one or more transgenic organisms;

Providing a gene expression “profile” for studying the effects of abiotic, biotic, and synthetic stimuli on gene expression;

Studying gene expression spatially in the same organism (three dimensional gene expression);

Studying gene expression over time (temporal gene expression);

Studying gene expression as a function of development of a cell or organism; and

Studying the effect of mutations in coding or regulatory sequences on gene expression.

In one embodiment, organisms (e.g. plants or animals) or cells from organisms to be examined for gene expression are genetically transformed by introduction of a gene construct containing an OCM detectable reporter gene. Detection or measurement of the OCM signal resulting directly or indirectly from a product or products of the reporter gene, allows detection and analysis of the expression of the reporter gene. In addition to directing its expression in vivo, the substance may be introduced into the organism, tissue, organs, cells of study using methods well known to those skilled in the art (e.g., microinjection).

In another embodiment, OCM data generated from two or more biological samples are systems are analyzed and compared mathematically using histogram analysis of OCM binary data to detect differences in biological function (morphology, gene activity, cell development, and the like) between the samples. This method provides rapid and reproducible data comparisons that facilitate automated and high-hroughput analysis of OCM data from biological systems.

In yet another embodiment systems and methods for high throughput OCM analysis and measurement are provided that allows rapid and accurate comparisons of very large numbers of biological samples. More specifically, these methods allow rapid and accurate establishment of a normal dataset (a ‘profile’) for a given biological system and comparison of a large number of individual samples to that normal dataset to detect individuals that are different from the normal.

Optical Coherence Microscopy

Optical coherence microscopy provides high resolution cross-sectional images of biological tissues by measuring light that is backscattered from the tissue sample. In general, OCM signal is a property of photon reflectance caused by local variation in indices of refraction. Because organelles and molecules (e.g. lipid versus water) have different refractive indices they produce distinct amounts of OCM signal. In one embodiment, the OCM image is false-colored, wherein red=most signal; blue=least signal, black=no signal. The OCM signals from typical cell structures have been theoretically and experimentally analyzed (Drezek, R., et al. (1999) Applied Optics 38, 3651-3661; Dunn, A., and Richards-Kotum, R. (1996). IEEE J. of Selected Topics in Quantum Electronics 2, 898-905.) The OCM signal is greatest from nuclei, somewhat less from organelles (e.g. mitochondria and chloroplasts), less from rough endoplasmic reticulum and least from the cytoplasm. In one embodiment, the system of this invention is set so that air and water will have a zero value. Hence, normal OCM images of plants typically have nuclei false-colored red, organelles and dense membrane systems yellow, cytoplasm green-blue. Vacuoles produce little or no signal and hence are colored black.

Detailed optical principles for OCM and the specifics of OCM instrumentation have been described previously (Fercher, A. F. (1996) J Biomed Optics 1, 157-173; Hettinger, J. W., et al., (2000), Plant Physiology 123, 3-15; Hoeling, B. M., et al., (2000). Optics Express 6:136-146; Hoeling, B. M., et al., (2000) Review of Scientific Instruments 72, 1630-1633; and Medford, J. I. et al., (2000) PCT Application Number WO 00/45153, all incorporated herein in their entirety). A brief description of the data collection and visualization methods of this invention follows.

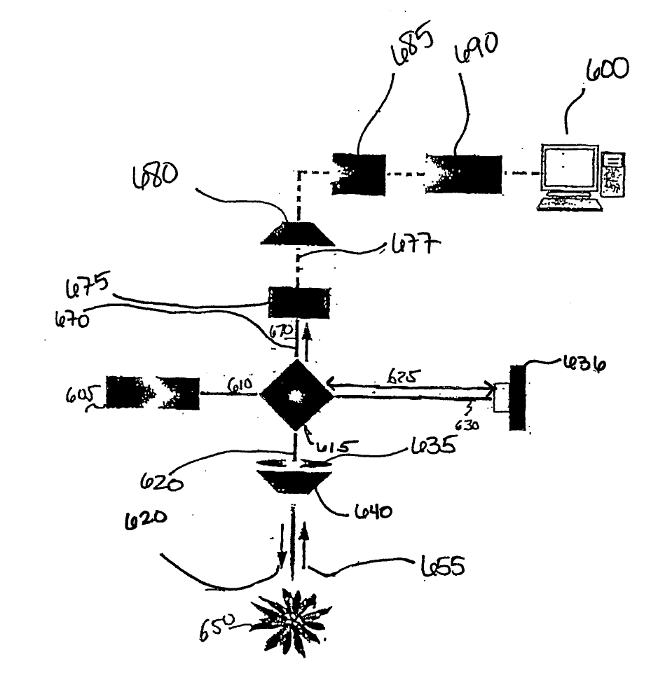

FIG. 1 shows a system 600 for acquiring and using OCM data for a sample, according to one embodiment of the present invention. A light generator 605, such as a light emitting diode, is used to generate light of a particular suitable frequency. The light may be visible, near infrared, infrared, or light of another frequency. The light is conveyed using a light transmission medium 610, such as an optical fiber, a waveguide, a vacuum, or a carrier gas like air. A beamsplitter 615 splits the generated light into a first light beam, known as an incident light beam 620 that is directed toward the sample 650. A second light beam, known as a reference beam 630 is used as a reference to the incident light beam for comparison or other purposes. According to one embodiment delay may be introduced into the reference beam, such as having the beam transmitted a given length 625 of a transmission medium. The reference beam may then be reflected back through the transmission medium using the functionality of a motorized reference mirror 636 or other device.

The incident light 620 may be processed by components like the motorized scanning mirrors 635 and focusing lens 640 that may be used to manage and control the location of the incident light 620 on the sample 650. Alternatively, the sample could be repositioned using a sample moving apparatus. The light may be focused on a portion of the sample, such as a surface plane or a voxel. The term ‘voxel’ is used herein to refer to a volume element of the sample, having an arbitrary, but frequently predetermined size and shape. A voxel is also described in the art as a 3-dimensional pixel. Voxel size may depend on the size of the structures within the sample that are being visualized or analyzed. For example, smaller voxels may be used for some biological structures.

Back-scattered light 655 may or may not be focused or otherwise processed by components used to process or focus the incident light 620. The back-scattered light from the sample interferes is combined with the delayed reference light beam 630 and interferes with coherently with the reference beam only when the optical path lengths for the two are equal within about one coherent length of the light source. Scattered light from other sample depths is substantially excluded using this coherence method. The recombined beam 670 is processed by a light intensity detector 675, such as a photodetector, that may typically generate an electrical signal based on properties of the light 670, such as intensity, and frequency or color. The electrical signal 677 may be amplified by an amplifier 680 and filtered by an electrical filter 690, such as a circuit. Alternatively, filtering and amplifying could be done in software. The electrical signal, after any optional processing, may be processed by a device 690 for converting the electrical signal into computer-readable format, such as a binary format. An RMS voltmeter may be used according to one embodiment. The computer-readable data may then be processed and analyzed on computer system 695. Optionally, the data may be displayed as a visual representation of the structural features of the sample.

An exemplary optical coherence microscope, including exemplary methods and descriptions of optical components and combinations, light sources, scanning techniques, beam focusing, improvements of image acquisition time, OCM image interpretation, OCM calibration, high frequency modulation of path length, and enhancements to OCM design and use, among other topics, are discussed in detail in PCT publication number WO 00/45153, incorporated herein in its entirety, to the extent not inconsistent herewith. While the OCM system and methods discussed in WO 00/45153 are exemplary, it will be obvious to one skilled in the art that the methods for gene expression analysis of this invention may be practiced with other OCM/OCT systems known in the art.

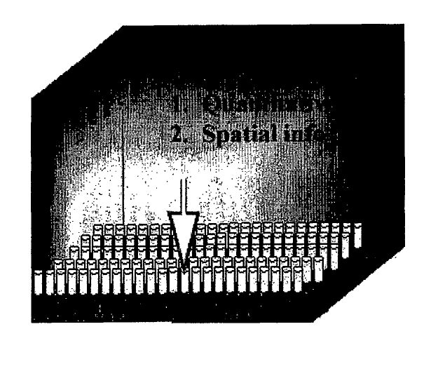

In a specific embodiment of this invention, the light source for the instrument is a near-infrared light at 850 nm provided by a very low intensity (300 μW) superluminescent diode (SLD). The light beam travels along single-mode optical fibers. The light is split (50/50) with half going along the sample arm to the plant and half going along the reference arm to a mirror. The light is focused to a small 10×5 μm voxel. Photons from a voxel at a specified depth (z-plane) are collected in approximately three microseconds. The light is moved to the next voxel in the transverse direction (x and y) with rotating mirrors and a scan of a given plane typically collected in three seconds (FIG. 2). After each scan of a plane, the focusing lens is stepped down in depth and another plane is scanned. Hence, data are three-dimensional and directly represent three dimensional aspects of the living organisms.

Signals from the OCM are stored in computer memory as a three-dimensional data set with the position of each voxel defined. Hence, OCM assembles a true three-dimensional image from smaller, three-dimensional components (FIG. 2) Because of this, OCM images can be disassembled as desired for analysis. This is substantially different from “3D reconstructions”, such as those done with a confocal data. A 3D reconstruction is simply an assembled series of flat, 2D planes. Because of this, the 3D reconstructions can only be disassembled in one plane, potentially obscuring three-dimensional features. OCM collects true three-dimensional images that can be cropped and/or optical slices prepared in any plane. In addition, the optical slice, cropped volume or entire volume can be rotated in any plane or re-sized and the colormap adjusted (in a linear manner) to distinguish features of interest. Also opacity can be adjusted, for example, to make an optically isolated organ (e.g., leaf primordium) “see-through”. Each voxel can also be analyzed individually.

Analysis of OCM Data

This invention further provides multiple novel data analysis methods for the interpretation of OCM data. In addition, the invention provides methods to accelerate data acquisition, interpretation, and utilization, making the OCM gene expression analysis system useful as a high-throughput system for gene expression detection and measurement and for screening of genomic databases.

OCM datasets are typically composed of 1-10 million voxels with each voxel having a voltage value between 0-10,000 mV. The recorded voltage (or other signal unit) for each voxel is herein referred to as the OCM binary data. The system of the present invention has a low level, approximately 30-50 mV, of electrical noise. Scans of 1×1×1 mm³ can be completed in one minute with a digital processor. The resolution of the images is 10×5 um. Image recognition can be greatly improved by repeated voxel sampling and structures that are approximately 2 um in size can be visualized (see below). Voxels of specific values within OCM datasets are false-colored and 3D images produced using scientific software for 3D visualization, AVS Express (Advanced Visualization Systems, Waltham Mass.). The data acquired in an OCM dataset further comprises spatial information that allows 3-dimensional imaging.

As described above, the OCM data are initially in a binary format. To develop the ability to use OCM in a high throughput system, and the ability to image and analyze gene expression, novel methods are utilized to analyze the binary data prior to its visualization. These methods are embodied in a machine readable format as detailed in U.S. patent application Ser. No. 60/264,641, incorporated herein in its entirety, and serve four main purposes. First, methods are included that allow reduction or elimination of electrical noise from the OCM data. Second, OCM data is converted for export into standard graphing or spreadsheet programs (e.g. Excel, DeltaGraph) for examination by histogram analysis. Third, the ABAS software allows separation of the endogenous OCM signal from the signal that is due to differences in biological function, including but not limited to transgene activity. This isolated signal is herein referred to as “gene reporter” signal (see below). Fourth, the binary data, before or after histogram analysis, is converted to a format compatible with visualization software.

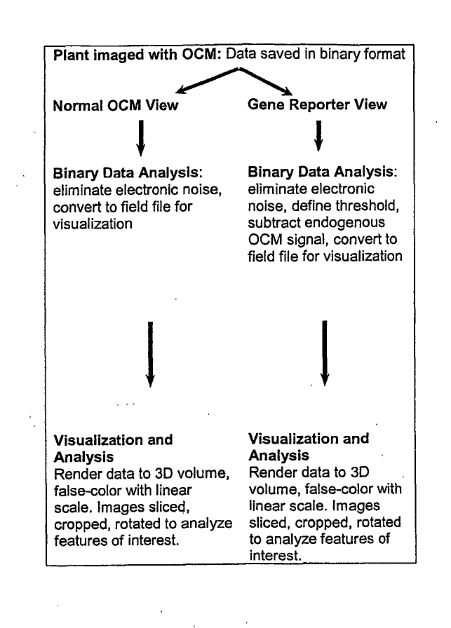

FIG. 3 is a flow chart illustrating one example of the steps taken to visualize endogenous plant or animal structural features or biological function (e.g. gene activity or expression, growth, and the like). The present invention allows the same data to be visualized as either the “normal OCM view” which allows endogenous features of the plant or animal to be visualized or the “gene reporter view” which excludes endogenous OCM signal due to, for example, cell structure. This allows the study of how, for example, gene expression is altered in the same plant or animal that is being studied. The alteration in gene expression can be due to, for example, response to a biotic, abiotic, or synthetic stimulus or stimuli. Such stimuli are well known in the art, and include pharmaceutical compounds, toxins, light, wind, moisture, and the like.

Comparing images individually with the visualization software is an accurate, but slow and laborious method. In a novel aspect of this invention, histogram analysis of the OCM data is employed to quantitatively represent the OCM data and automate sample comparison. As described above, a photodetector in the OCM converts the number of photons per voxel to a voltage. The 3-dimensional dataset for each sample is collected by a computer in binary format as described above. Also as described above, the binary OCM data can be exported to standard graphing programs to prepare a histogram of the data.

Histogram analysis is used in one embodiment of this invention to compare two or more biological samples, such as tissues, cells, organs, and organisms. The samples may represent wild-type, or ‘normal;’ samples, or samples that have been altered (e.g. genetically transformed), samples at different developmental stages, or samples that have been exposed to biotic, abiotic, synthetic or environmental stimuli or toxins. In a simple embodiment, OCM for each sample is collected and converted to a histogram. The histograms are compared visually or mathematically (e.g. subtraction) to determine if differences exist between the samples. As discussed above, it may also be desirable to convert ‘aberrant’ histograms back to a 3-dimensional representation to try to determine visually how that sample is different.

Previous studies examining various OCM images from hundreds of samples indicated samples of the same age are very similar. The similarity of OCM images of plants is consistent with other studies reporting, for example, that measurements with OCM/OCT were very consistent for the retinal nerve fiber layers. See Blumenthal et. al., Ophthalmology, 107(12): 2278-82 (2000). Hence, by examining numerous samples of the same structures, tissues and age, variations can be averaged and a “profile” of the OCM signal for a given sample produced. In addition to creating “profiles” of the OCM signal from simple tissues such as cotyledons, OCM profiles can also be created for tissues that are rapidly changing, for example the Arabidopsis shoot apex.

In another embodiment, endogenous OCM data is obtained for a number of non-transgenic reference samples or another group of well-characterized reference samples having similar OCM data sets. The data for each individual reference is displayed as a histogram: number of voxels per signal unit (mV). The histogram categories, then, correspond to millivolt values. The average number of values for each histogram category for the nontransgenic organisms is determined as well as the standard deviation for each histogram category. These averaged histograms constitute a baseline reference, or normal ‘profile’, to which other organisms or cells are compared.

Next, OCM data for a sample organism, tissue, organ or cell of interest is collected for comparison. This sample may be transgenic, another wild type sample, or either type that has been subject to a biotic, abiotic, synthetic, or environmental stimulus or stimuli. The binary data for the sample is presented in histogram form and compared to the endogenous baseline histogram of the wild type or ‘normal’ sample. This histogram analysis, or comparison, can be any mathematical manipulation (e.g. subtraction of the profile histogram from the sample histogram) of the two sets of binary data such that any difference between the data sets is apparent.

For example, if, for a particular histogram category, the number of voxels having a particular signal value falls within a certain range (e.g. the endogenous mean plus or minus one standard deviation), those voxels are set to zero in the resulting compared data set (the “gene reporter” data set). If, on the other hand, the number of voxels for a given signal value is outside the range for the endogenous dataset, this is reflective of a difference in biological activity such as developmental activity, transgene activity, growth, and the like. All voxels of that value are assigned a color representing their signal value in the resulting “gene reporter” dataset. Because the binary data of the ‘gene reporter’ dataset retains 3-D information for each voxel (i.e. the 3D location for each voxel), the binary data may be converted to a 3-D representation, even after histogram analysis and mathematical manipulation of the signal data. Thus, the “gene reporter” dataset, when visualized, produces images in which OCM signal attributable to endogenous cell sources is effectively removed. This image view is referred to as the “gene reporter view”.

In still other embodiments, it may be useful and desirable to analyze only a particular signal range of the OCM data by establishing a minimum and/or maximum threshold of signal values, either prior to histogram analysis or conversion of the binary data to its 3-dimensional representation or image. Signal values below the minimum threshold are set to zero, while values above the maximum threshold are set either to the maximum value in the dataset or to zero. Value within the threshold may be treated further, such as with the application of a color-gradient to the remaining signal range to aid in viewing the three dimensional representation.

The methods of this invention also utilize additional methods of image analysis to distinguish between signal due to gene activity or other biological functions and exogenous signal. In general, the OCM response at a voxel is a combination of the density of the OCM detectable substance and the density of the background structure. It is therefore possible to detect very low levels of OCM detectable substance by first classifying voxels by their type, where the type of a voxel (nucleus, organelle, etc.) is determined not only by the OCM response of the voxel, but also on the geometry of the responses of neighboring voxels. This type of analysis is typical of computer vision and pattern recognition research, and methods relating to this type of analysis are readily known to those skilled in the arts of computer vision, computer science, and other data analysis fields.

These methods for analysis of OCM data using histogram analysis of the binary data are representative only. It will be apparent to one of ordinary skill in the art that there are numerous analytical situations in which the comparison of one or more samples to another or to groups of samples will be facilitated by analyzing binary data as described in this invention.

Analysis of Gene Expression with OCM

In several specific embodiments the methods of this invention employ gene constructs for the introduction of an OCM detectable reporter gene into a biological system to assess gene expression in that system. Other methods of this invention can be employed to assess gene expression in transgenic biological systems. In general methods for making gene constructs and for introducing gene constructs into cells, tissue, organs, or organisms to contain and express the coding sequences in the gene constructs are well known in the art. Once the gene construct is introduced into host cells or organisms, the presence of such construct can be verified by a variety of art-known methods, i.e., PCR, Southern hybridization, and selection by the addition of a drug. The gene constructs useful for practicing the claimed invention include but are not limited to those which contain a coding sequence for a gene product detectable by OCM with endogenous or exogenous regulatory sequences.

For the Purpose of the Present Invention, the Following Terms are Defined:

“Gene” is a nucleic acid sequence that includes the transcribed sequence encoding a mRNA, a sequence that encodes a protein (including “exons”) along with any associated regulatory elements (whether upstream or downstream of the coding sequences) to which the coding sequences are operably linked and optionally any untranslated intervening sequences (“introns”) that may be associated with a given gene.

“Gene construct” refers to a nucleic acid molecule that contains one or more coding sequences, for example a gene or genes of interest and/or a reporter gene, one or more regulatory sequence or regulatory elements for expressing the gene of interest and/or the reporter gene. A gene construct may also contain one or more selectable marker genes, in addition to the reporter gene, to facilitate selection of host cells, tissue, or organisms that contain the gene construct. A gene construct may also contain a replicon for propagation in a host cell. A gene construct may also include an entire synthetically produced group of regulatory elements, coding sequence and the like.

A recombinant vector or an expression vector is one example of a unit that contains a gene construct. A recombinant vector gene construct of this invention comprises an OCM-detectable reporter gene or another gene of interest inserted into any vector capable of delivering the gene(s) into a host cell, tissue, or organism and maintaining it. The vector can be either RNA or DNA, prokaryotic, eukaryotic or synthetic in origin, and typically is a virus or a plasmid, although any form of recombinant vector or expression vector may be used in the practice of the present invention. Recombinant vectors can be used in various cloning, sequencing, and/or other manipulation of the nucleic acid molecules of the present invention. In addition to recombinant vectors, other forms of nucleic acid molecules and/or proteins and/or synthetic combinatory molecules may be used to direct the expression of an OCM detectable reporter gene.

More specifically, an expression vector comprises a coding sequence of interest operatively linked to one or more regulatory sequences or elements effecting expression of the particular sequence of interest. Preferably, the expression vector is also capable of replicating within the host cell. The expression vector can be a DNA or RNA vector, either prokaryotic or eukaryotic of origin, and is typically a virus or plasmid. Expression vectors of the present invention include any vectors that function (e.g. direct OCM-detectable gene expression) in a selected host, including bacterial, yeast, fungal, endoparasite, insect, animal or plant cells. Commercially available expression vectors can be used. Vectors can include host-recognized replication systems, amplifiable genes, selectable markers, host sequences useful for insertion into the host genome, and the like, as well as at least one gene of interest or at least one gene whose expression results in an OCM-detectable product.

Viral vectors are well known in the art and may be employed as gene constructs in the present invention. Vectors derived from viruses such as vaccinia virus, adeno-associated virus (AAV), and herpes viruses may be employed. They offer several attractive features for use in various mammalian cells. Commercially available baculovirus vectors can be used in cultured insect cells or insects.

In preferred embodiments, the gene constructs are suitable for expression in a host plant or animal of interest or cells or tissues thereof.

“Gene Expression” includes synthesis of a product of gene transcription or translation including but not limited to protein synthesis, trans-splicing, RNA editing, the post-transcriptional or post-translational modification thereof, and formation of structures or products resulting from functional activity or said transcription and translation products as is well known to those skilled in the art. The term “gene expression” also includes the synthesis of a product that results from the action of a plurality of genes.

“Reporter gene” is a gene containing a nucleic acid sequence encoding (i.e. a coding sequence) a gene product which is detectable and which can be employed to assess gene expression levels. The coding sequence of the reporter gene is operably linked to and under the regulatory control of any selected regulatory sequences and may be under the regulatory control of regulatory sequences of one or more genes of interest.

An OCM detectable substance is any material produced within or introduced into a cell which can form or cause formation or degradation of particles, aggregates, inclusion bodies and the like that are substantially opaque and back scatter light at the wavelength of light used for the OCM measurement. The characteristics of OCM detectable substances can be readily determined by-one skilled in the art based on known optical principles and theory of light scattering and back scattering. As an example, the preferred OCM detectable substance described herein, PHB is an opaque granular substance of about 200 to about 700 nm in size.

An OCM-detectable reporter gene is one whose expression affects the presence of an OCM-detectable substance. In specific embodiments herein an OCM-detectable reporter gene is employed which comprises at least one coding sequences whose expression contributes to the production or degradation of a substance that is detectable with optical coherence microscopy (OCM), i.e., a substance whose OCM signal can be differentiated from the endogenous OCM signal of living organisms. Any gene whose product may be imaged by optical coherence microscopy can be used as the OCM-detectable reporter in the methods of this invention. Any gene or combination of genes whose product or products contribute to the synthesis, modification or degradation of an OCM-detectable substance are within the scope of this invention and can be used as the OCM-detectable reporter gene in the methods of this invention. In a specific embodiment, a gene whose product (e.g., a biosynthetic enzyme) contributes to the synthesis or degradation of a biopolymer, such as PHB, can be used as an OCM-detectable reporter gene in the methods of this invention. Non-limiting examples of genes useful as OCM-detectable reporter genes include genes involved in glycogen synthesis or degradation, genes involved in the production of protein crystals (for example, Bacillus thuringiensis crystal protein), genes involved in lipid production or degradation and genes involved in production or degradation of synthetic or naturally occurring polymers. It will be appreciated that in cases in which an OCM-detectable product is generated indirectly by the action of more than one biosynthetic enzymes that genes encoding all of the required biosynthetic enzymes may be required to obtain production of the desired OCM-detectable product. It may in some cases be necessary or desirable to provide (or increase the level of) a starting or intermediate substrate for production of the OCM-detectable substance to the biological system containing the OCM-detectable reporter gene. Imaging using the OCM-detectable reporter gene(s) may in certain cases be enhanced by operably linking a targeting, transit or signal peptide sequence to the coding sequence that allows for localization of a gene product in one or more subcellular compartments. Such sequences and their use in gene constructs are well known in the art.

“Regulatory sequences or elements” include any nucleic acid sequence or sequence elements that effect the level, timing, site or stability of expression of a coding sequence in in vivo, ex vivo or in vitro biological, transcription, translation and/or expression systems, such as a cell. Regulatory sequences include transcription control sequences, translation control sequences, origins of replication, and other regulatory sequences that are compatible with a host cell and that control the expression of coding sequences of interest in the host cell. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the chosen hosts of this invention. A variety of such transcription control sequences include those which function in bacterial, yeast, plant, insect and mammalian cells, such as, but not limited to, 35S, β-actin, myogen, alcohol dehydrogenase, nos, tubulin, tac, lac, trp, trc, oxy-pro, omp/Ipp, rrnB, bacteriophage lambda (such as lambda P_{L} and lambda P_{R} and fusions that include such promoters), bacteriophage T7, T7lac, bacteriophage T3, bacteriophage SP6, bacteriophage SP01, metallothionein, alpha-mating factor, Pichia alcohol oxidase, alphavirus subgenomic promoters (such as Sindbis virus subgenomic promoters), antibiotic resistance gene, baculovirus, Heliothis zea insect virus, vaccinia virus, herpes virus, raccoon poxvirus, other poxvirus, adenovirus, cytomegalovirus (such as intermediate early promoters), simian virus 40, retrovirus, actin, retroviral long terminal repeat, Rous sarcoma virus, heat shock, phosphate and nitrate transcription control sequences as well as other sequences capable of controlling gene expression in prokaryotic or eukaryotic cells. Additional suitable transcription control sequences include tissue-specific promoters and enhancers as well as lymphokine-inducible promoters (e.g., promoters inducible by interferons or interleukins). Transcription control sequences can also include naturally-occurring or synthetic transcription control sequences found in humans.

Regulatory sequences also include but are not limited to introns, signal sequences, leader sequences and other elements well known in the art. As is known in the art regulatory elements may function alone or in concert with other regulatory elements. The spacing and or relative positioning of regulatory elements with respect to each other and or with respect to a coding sequence can affect their function.

Gene constructs may be optimized for expression of coding sequences in a given host. Optimization can occur, for example, by selection of regulatory elements, by positioning and spacing of regulatory elements, by substitution of preferred codons selected for expression in the host plant or animal cell of interest in one or more coding sequences in the constructs or combinations of these techniques which are all well-known in the art.

Introduction of a gene construct into a cell can be accomplished by any method known in the art. The means of introducing the gene construct into a host cell varies depending upon the particular construct and the host. The host containing the gene construct is designated a recombinant host or a transformed host. Suitable means for introduction of gene constructs include fusion, conjugation, transfection, transduction, electroporation, lipofection, adsorption or microinjection, as described in Sambrook, supra, for example. Additionally, plants and plant cells may also be transformed viaAgrobacterium-mediated transformation, electroporation, vacuum infiltration or a particle gun. See Bechtold et. al. (1993) C.R. Acad. Sci. Paris, Sciences de la vie, 316:1194-1199. A wide variety of host cells can be employed for expression of the gene construct both prokaryotic and eukaryotic. A preferred plant cell for transformation is Arabidopsis thaliana. See Nawrath et. al. (1994) PNAS 91:12760-12764 (1994). A recombinant (or transformed) cell containing a gene construct may remain unicellular or may grow, from or be regenerated into a tissue, organ or a multicellular organism. Alternatively, a recombinant or transformed cell may differentiate into another cell type. Alternatively, transformed tissue or cells may dedifferentiate. Nuclei acid molecules, proteins and other molecules can also be directly introduced into a cell or organism for study (e.g., micro-injection). As known in the art, transformed nucleic acid molecules of the present invention can remain extrachromasomal or can integrate into one or more sites within a chromosome of the transformed (i.e., recombinant) cell in such a manner that their ability to be expressed is retained.

Several other methods for the transfer of gene constructs for production of OCM-detectable products or other gene products of interest into cultured mammalian cells also are contemplated. These include, but are not-limited to, calcium phosphate precipitation (Graham and Van Der Eb, 1973; Chen and Okayama, 1987; Rippe et al., 1990) DEAE-dextran (Gopal, 1985), lipofectamine-DNA complexes, and receptor-mediated transfection (Wu and Wu, 1987; Wu and Wu, 1988). Some of these techniques may be successfully adapted for in vivo or ex vivo use.

The gene construct of this invention may simply consist of naked recombinant vector. Transfer of the construct may be performed by any of the methods mentioned above which physically or chemically permeabilize the cell membrane. For example, Dubensky et al. (1984) successfully injected polyomavirus DNA in the form of CaPO₄ precipitates into liver and spleen of adult and newbom mice demonstrating active viral replication and acute infection. Benvenisty and Neshif (1986) also demonstrated that direct intraperitoneal injection of CaPO₄ precipitated plasmids results in expression of the transfected genes in animal models.

Recombinant DNA technologies can be used to improve expression of transformed nucleic acid molecules by manipulating, for example, the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of a coding sequence of interest include, but are not limited to, operatively linking nucleic acid molecules to high-copy number plasmids, integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of nucleic acid molecules of the present invention to correspond to the codon usage of the host cell, deletion of sequences that destabilize transcripts, and use of control signals that temporally separate recombinant cell growth from recombinant enzyme production during fermentation. The activity of an expressed recombinant protein may be improved by fragmenting, modifying or derivatizing nucleic acid molecules encoding such protein.

“Host” when used in reference to a cell, tissue, or organism refers to a cell, tissue or organism that is transformed to contain a gene construct and which preferably expresses a coding sequence of the construct under the control of endogenous or exogenous regulatory elements in the gene construct or endogenous regulatory elements native to the cell, tissue or organism. Suitable host cells to transform include any cell that can be transformed with a nucleic acid molecule as described herein. Host cells can be either untransformed cells or cells that are already transformed with at least one nucleic acid molecule (e.g., nucleic acid molecules encoding one or more proteins of the present invention and/or other proteins of interest or in the production of PHB or other polymeric material of interest.) Host cells include bacteria, such as E. coli, yeast, filamentous fungi, insect cells, plant cells, mammalian cells, which may be immortalized, e.g., mouse, CHO, human and monkey cell lines and derivatives thereof. It may or may not be preferable for the host cells to process the OCM-detectable reporter gene (and/or other genes of interest) to produce an appropriate mature polypeptide. Processing can include glycosylation, ubiquitination, disulfide bond formation, general post-translational modification, and the like. Host cells can be any cell capable of producing at least one protein or OCM-detectable product of interest, and include bacterial, fungal (including yeast), insect, animal and plant cells, such as Salmonella, Escherichia, Bacillus, Listeria, Saccharomyces, Spodoptera; Mycobacteria, Trichoplusia, BHK (baby hamster kidney) cells, COS (e.g., Cos-7 cells and Vero cells) and plants such as Nicotiana and Arabidopsis, among others.

Endogenous as used in reference to nucleic acid sequences refers to those sequences that derive from the host.

Exogenous as used in reference to nucleic acid sequences refers to those sequences that do not derive from the host and/or are man-made.

Synthetic, as used in reference to nucleic acid sequences, refers to those sequences that do not derive from the host and/or are man-made.

While a variety of OCM-detectable reporter genes may be used within the scope of the present invention, preferred genes include those encoding enzymes whose products include plastics such as poly-3-hydroxybutyrate (PHB) or other polymers as well as genes encoding proteins involved in formation of abberant proteasomes and other inclusion bodies. Both the PHB system and the proteasome system cause the formation of opaque granules that are at least about 200 nanometers-in size, in biological systems. Hence, this invention covers a variety of gene products (e.g., other plastics, other inclusion bodies) that would have similar properties. It will be understood by one skilled in the art that improvements in OCM technology can expand the range of OCM detectable substances available for use with this invention. Most preferred are the three genes which encode the three enzymes necessary to catalyze synthesis of poly(R)-(3)-hydroxybutyrate (PHB) from acetyl-CoA. PHB has been shown to produce significant a OCM signal. These genes may be modified for expression in plants by addition of DNA encoding an appropriate transit peptide (e.g., the pea chloroplast) and appropriate polyadenylation sequence. See Nawrath et. al., Targeting of the polyhydroxybutyrate biosynthetic pathway to the plastids of Arabidopsis thaliana results in high levels of polymer accumulation,” Proc. Natl. Acad. Sci. USA 91:12760-12764 (1994).

PHB is a natural, biodegradable thermoplastic with chemical and physical properties similar to polypropylene. Accumulation of PHB in plant chloroplasts produces no detrimental effects with expression from the CaMV 35S promoter, a strong constitutive promoter, (up to 1% of the plant's fresh weight). [Poirier, 1993 #854; Poirier, 1995 #855; Poirier, 1995 #883; Bohmert, 2000 #839; Bohmert, 2000 #879] Thus, PHB is compatible for use in living cells.

The function of PHB as an OCM detectable product of gene expression was assessed in transgenic plants that were genetically engineered to produce PHB in their plastids. See Nawrath et. al., [Nawrath, 1994 #547]. PHB production in these plants is under control of the CaMV 35S promoter (a strong constitutive promoter) and directed to the plastids using the Rubisco small subunit leader. PHB accumulates as small granules (0.2-0.7 um in size) in the plastid stroma. See Bohmert et. al. (2000) Planta, 211(6): 841-5 (2000).

Three genes are required for PHB production in plants: phbA (3-ketothiolase), phbB (acetoacetyl-CoA reductase), phbc (PHB synthase). See Nawrath et. al., (1994). Without phbC, there is no accumulation of plastic. To produce a system that can be used to visualize gene expression in vivo, a stable transformant may be produced that has the phbA and phbB genes constitutively expressed. The phbC gene can then be introduced into the transformant under the control of a selected promoter (including a transcriptional promoter of interest) to follow expression patterns in vivo. PHB production can also be directed, somewhat inefficiently, without the phbA (3-ketothiolase) gene. Plant transformations can use the well-characterized vacuum infiltration method. See Bechtold et. al., (1993). This method is capable of producing hundreds to thousands of transgenic plants from one transformation experiment.

In one embodiment of this invention, transgenic OCM gene reporter lines can be produced for use in the methods of this invention by placing the phbA and phbB genes in tandem on a single T-DNA under the control of the CaMV 35S promoter and introducing the construct into plants. Plants that transcribe both the phbA and the phbB genes at high levels are detected, e.g., using RT-PCR. Plants with high levels of both genes are selected and made homozygous. Tissue specific promoters and/or inducible promoters, such as the XVE promoter, are fused to the phbC gene to function as the OCM-detectable reporter gene (in a cell expressing phbA and phb.) The phbC fusions are introduced into lines homozygous for phbA and phbB by a second transformation event. The expression of the phbC reporter gene (with RT-PCR) and production of PHB (with a Nile Blue Assay) can be tested in the transgenic plants. A suitable plant host for assessing plant gene expression is Arabidopsis thaliana.

Those skilled in the art will also recognize that PHB degradation systems have been described indicating that the gene imaging system here could be employed to with a “gene reporter” that is capable of both induction and turnover [Saegusa, 2001 #908]. For example, the gene for degradation enzyme could be fused to a promoter or other regulatory molecule of interest, allowing study of genes with rapid turnover and study of plant, animal and human response to a variety of biotic, abiotic and synthetic stimuli. See Saito et. al. (1989) J. Bact., 171(1):184-189 (1989).

The invention can, for example, be employed to assess expression in transgenic plant and animal and human tissues. Production of transgenic plant and animals is well known to those skilled in the art. For example, U.S. Pat. Nos. 5,484,956 and 5,538,880, provide a method for creating fertile transgenic Zea mays; U.S. Pat. No. 5,489,520, provides a method of creating stable, genetically transformed maize cells and methods of selecting cells that have been transformed; Hogan et al., “Manipulating the Mouse Embryo, A Laboratory Manual,” Cold Spring Harbor Laboratory. Inactivation of endogenous variant genes can be achieved by forming a transgene in which a cloned variant gene is inactivated by insertion of a positive selection marker. See Capecchi, Science 244, 1288-1292 (1989). The transgene is then introduced into an embryonic stem cell, where it undergoes homologous recombination with an endogenous variant gene. Mice and other rodents are preferred animals. Such animals provide useful drug screening systems.

Additionally, once transgenic plants containing gene expression patterns are identified, the OCM imaging system can be used to follow how these genes are expressed in a single organism throughout development. In addition, the system could be used to follow how a class of genes responds to various biotic or abiotic stresses or pharmaceutical compounds.

The methods of the present invention can be employed to assess expression of mutant genes, particularly of genes under the control of regulatory sequences that have been mutated. Mutants can be prepared by any method known to those skilled in the art. Examples of mutagenesis techniques include but are not limited to ethyl methyl sulfate mutagenesis, site directed mutagenesis, insertional mutagenesis, deletional mutagenesis, gene replacement mutagenesis, recombinational mutagensis among others.

High-throughput OCM System:

As indicated above, the in vivo imaging technology can be applied to a high throughput system for the analysis of a plurality of samples. The OCM takes approximately one minute per scan. A high throughput system can be constructed with robotics and a CCD camera to locate and position the samples. The following is an example of the high throughput system that could be constructed with our invention. It takes, on average, one minute to position a sample under the OCM scanner. With 1440 minutes/day collection rates of at least 500 images per day are possible. Multiple OCMs per high-throughput system would improve that rate.

As an example of high-throughput gene expression analysis, the Arabidopsis genome contains 25,498 genes. This allows this number of plants to be screened for in vivo mutations in 51 days. Moreover, large sets of transgenic plants with random promoter fusions to the OCM reporter gene can be produced in a manner similar to that for destructive reporter genes ([Springer, 2000 #949; Martienssen, 1998 #863; Jefferson, 1987 #45]. Using OCM to measure gene expression in vivo, a scan of an initial database that represents 25,498 gene fusions (the size of the Arabidopsis genome) can be completed in a minimum of about 51 days. In comparison to prior art, genomic scale databases have typically taken years to construct [Venter, 2001 #944]. For a database with 95% probability of having the gene of interest tagged with a reporter gene, four-fold coverage is needed. A database of this can be completed in approximately 7 months using a single OCM system. The database can then be screened by computer for patterns of interest and all genes with specified spatial, temporal or response patterns rapidly identified, studied and/or mutagenized to identify regulators. The use of the automated high-hroughput OCM system of this invention can provide the first genomic-scale database of in vivo gene expression that includes temporal, spatial and expression level data.

Standard robotic system is readily adapted to the system of invention using technology well known to those skilled in the areas of engineering. In one embodiment robotics move a sample holder and identify the sample position for OCM imaging. As an example commercially available “Aratrays” can be used for samples of plants growing in soil. Other types of sample holders specific to cells, organs, organisms, tissues, or other types of plants will be readily recognized. The robotics described here incorporate technology to locate a sample to within 200 μm so the OCM can conduct the scan.

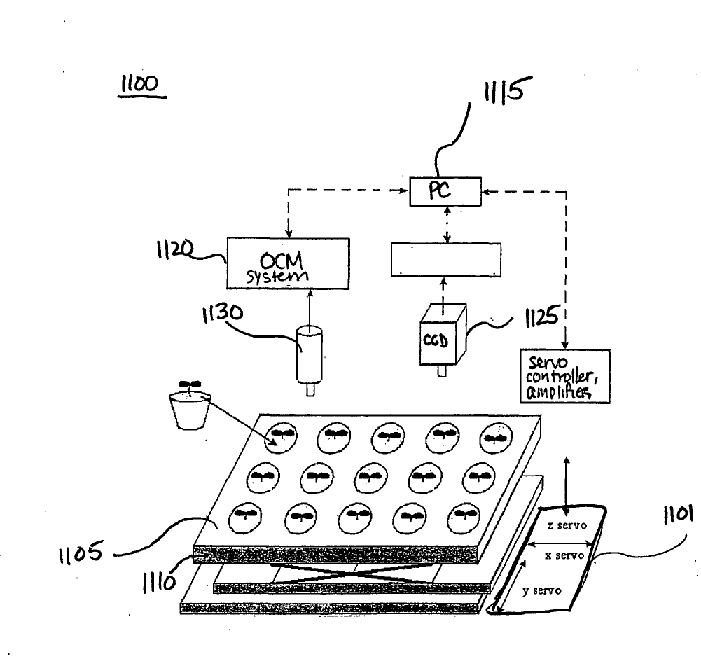

FIG. 4 illustrates the system that will be developed to provide high throughput automation using the Arabidopsis example. A flat of plants 1105 is held by a support frame 1110 and positioned accurately with a X-Y-Z servo motor-driven position table (1101). The servos will be controlled via an interface to a high performance personal computer (PC, 1115). The PC will also be interfaced to the OCM imaging system 1120 and a color CCD camera 1125. Images acquired from the CCD camera are used to identify and locate features on a plant or plant specimen so the features can be positioned accurately under OCM sensor 1130 for tissue imaging. The entire system is controlled by machine-readable code located on the PC.

The robotics system-functions as follows.

1. The x-y-z servo driven motion platform is first homed so the location of the flat is known relative to the fixed CCD camera and OCM sensor.

2. A specimen, identified by row and column numbers within the sample holder, is selected by the user automatically by the PC.

3. The motion platform is positioned to center a sample within the field of view (FOV) of the CCD camera.

4. An image is acquired (captured) from the color CCD camera and image processing is performed to precisely locate the sample or the desired feature on the sample. The first step of the image processing is to isolate the sample or sample feature from its surroundings. In the example of a plant, a threshold operation is performed on the green color component of the CCD image resulting in a simple binary image where the plant is white and everything else is black. Geometric features can be more easily identified and located. For example, the distal end of a leaf or the shoot apex can be located.

5. The coordinates of the center of the located feature are calculated and the motion platform is actuated to present the feature directly beneath the OCM.

6. The OCM is commanded to perform a scan and the computer stores the resulting voxel data.

7. The process continues back at step 2 for additional specimen.

Combining the Robotics and in Vivo Technologies

Genomic scale databases can be constructed by creating random gene-fusion libraries to plants with the OCM reporter gene (phbC and the −46 CaMV 35S promoter). This minimal promoter is placed near the right border of a T-DNA conferring resistance to BASTA or other types of selectable markers. Approximately 100,000 independent transgenic lines obtained to get 95% coverage of the Arabidopsis genome.

Screening using computerized methods can reduce gene expression patterns of 100,000 transgenic plants, to a few hundred. A human can screen a few hundred images identified in several days, yet have 95% confidence of complete genome coverage. The current methods to look at gene expression (gene expression profiling) on a large scale basis typically involves destructive techniques such as micro-arrays. Non-destructive techniques such as examination of GFP or GUS gene reporters must be done by hand and are extremely time consuming and can not be readily automated The automated robotics system of this invention would allow scientists to create computerized catalogs of gene expression that are three dimensional and include temporal, spatial, and quantitative information. Databases can be screened to follow how a class of genes respond to various biotic, abiotic or synthetic stimuli, including but not limited to responses to various pharmaceutical compounds. It will also be obvious to one skilled in the art to form other. gene-libraries and transgenic lines of other organisms, tissues, cells, and the like, which express and OCM detectable reporter gene. Such lines can be used with the method and materials of this invention for screening for response to a variety of abiotic, biotic and synthetic stimuli.

The present invention, as described above, includes various steps and methods. The steps of the present invention may be performed by hardware components or may be embodied in machine-executable instructions, which may be used to cause a general-purpose or special-purpose processor programmed with the instructions to perform the steps. Alternatively, the steps may be performed by a combination of hardware and software.

The present invention may be provided as a computer program product that may include a machine-readable medium having stored thereon instructions, which may be used to program a computer (or other electronic devices) to perform a process according to the present invention. The machine-readable medium may include, but is not limited to, floppy diskettes, optical disks, CD-ROMs, and magneto-optical disks, ROMs, RAMs, EPROMs, EEPROMs, magnetic or optical cards, flash-memory, or other type of media/machine-readable medium suitable for storing electronic instructions. Moreover, the present invention may also be downloaded as a computer program product, wherein the program may be transferred from a remote computer to a requesting computer by way of data signals embodied in a carrier wave or other propagation medium via a communication link (e.g., a modem or network connection). Accordingly, a carrier wave or other propagation medium shall be regarded as comprising a machine-readable medium for the purpose of the present specification.

### THE EXAMPLES

The following examples illustrate the present invention without, however, limiting it. It is to be noted that the Examples include a number of molecular biology, microbiology, immunology, imaging and biochemistry techniques considered known to those skilled in the art. Disclosure of such techniques can be found, for example, in Sambrook et al., ibid., and related references.

### Example I

### Analysis of PHB Expression in Transgenic Plants

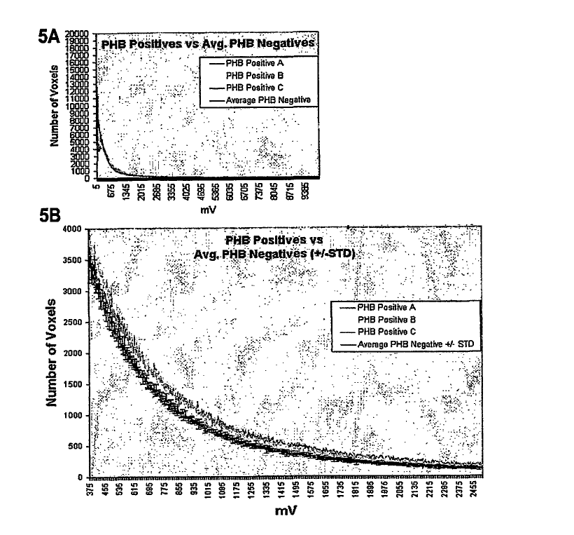

Arabidopsis cotyledons were imaged from wild type (Ws ecotype), transgenic PHB plants, and transgenic proteasome plants. The presence of each reporter gene molecule was verified with independent assays (Nile Blue assays for PHB, GFP tag for the proteasome). See Cutler et. al., (2000) and Poirier et. al. (1992) Science, 256:520-523. Analysis of OCM data as 3-dimensional images indicated that the proteasome and PHB plants produced more OCM signal than WT plants, Binary analysis was performed on the OCM datasets. FIG. 5A shows plots of the raw OCM data in binary format (number of voxels versus mV) over the entire signal range of the OCM. The plot for wild type plants is an average of 10 plants +/− one standard deviation. Consistent with the visualization of the transgenic lines, when the entire range of data is examined (0-10,000 mV) curves for wild type and PHB are similar, with slightly more signal apparent in the transgenic PHB line. However, FIG. 5B shows a plot of the raw OCM data examine over a limited range. The differences in the curves are accentuated.

Binary analysis methods of this invention were applied to the data generated from PHB transgenic and wild type Arabidopsis samples. FIG. 5B shows the histogram data for both over a limited signal range. To visualize the OCM signal from the transgenic PHB plants free of the endogenous OCM signal, high and low thresholds were set. The average signal (plus one standard deviation) of a wild type plant was subtracted from a transgenic plant. The comparison is done for each individual voxel and the output is then converted to a file appropriate for the visualization software. The resulting three dimensional image is referred to as the ‘reporter gene view’.

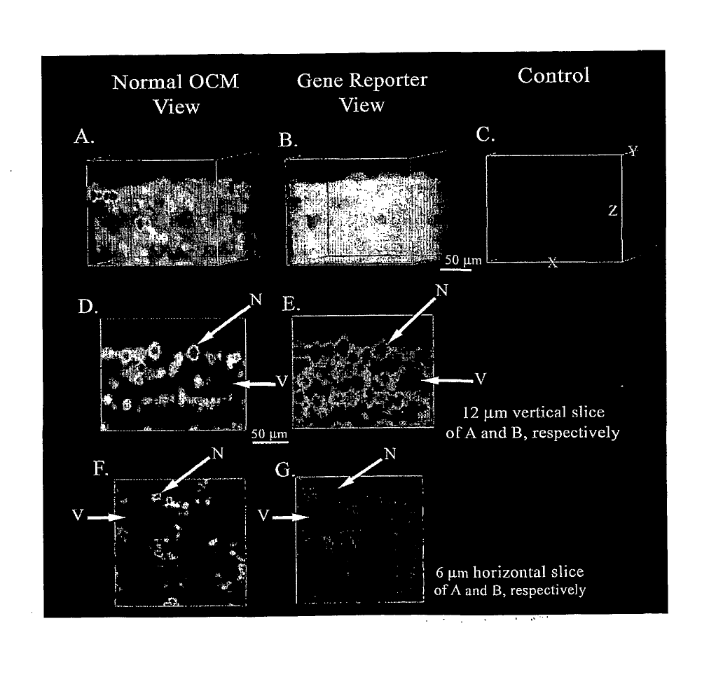

FIG. 6 shows an example of a “chunk” from an Arabidopsis cotyledon visualized in the “normal view” (complete OCM signal) and the same data visualized in the “gene reporter view” (PHB signal subtracted from the endogenous signal). Similar results are obtained for the proteasome line (also under control of the CaMV 35S promoter). For the PHB plants, gene expression is directed to the plastids. Hence, it is predicted that the PHB should accumulate in plastids, but not nuclei or vacuoles. FIG. 6D-G show a horizontal and vertical sections of the volumes in FIG. 6A-B. Signal is absent from the vacuole and nuclei as predicted. The “reporter gene signal” is seen in particulate regions that are at, or beneath, our level of recognition. These particulate regions are consistent with the plastids where PHB expression is directed.

### Example II

### Screening

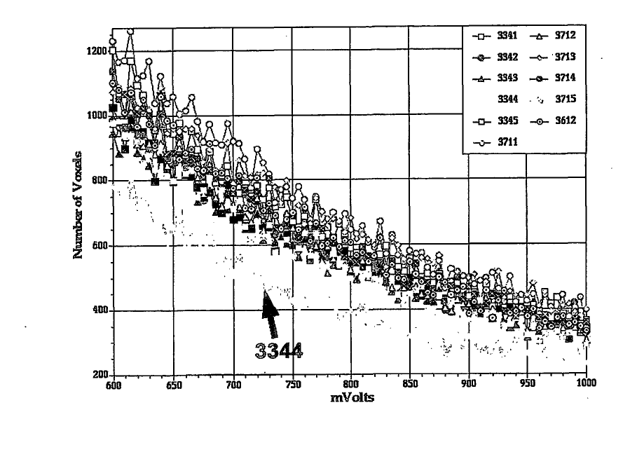

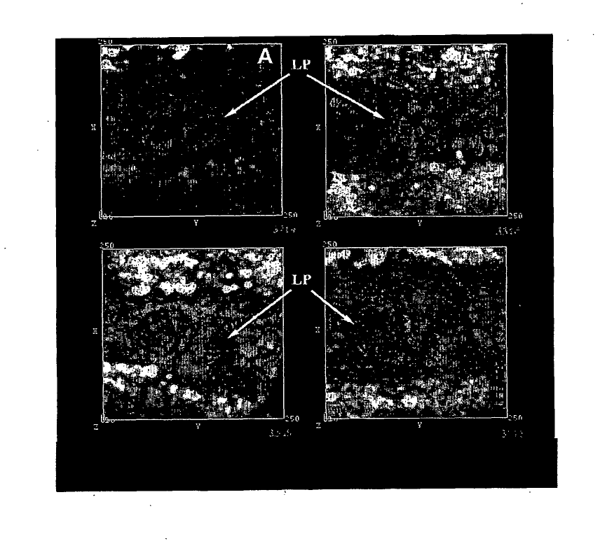

FIG. 7 shows histograms of OCM data acquired from eleven 6 day old Arabidopsis seedlings. The data are presented as number of voxels versus voltage. The histograms show that most OCM signals for plants of this age are very similar, with the exception of one plant (labeled 3344), whose OCM signal was distinctly different from others. Following histogram analysis, the binary data for plants 3344, 3714, 3344 and 3711 were converted to field files and visualization software was used to produce the images found in FIG. 8. The plant identified in the histogram as an outlier is shown in this figure to have aberrant leaf primordial. FIG. 8 also shows that even if a plant is scanned at a slightly different orientation (plant 3345), the change in orientation is not reflected in the histogram analysis as a difference in OCM signal.

The present invention is not to be limited by the preferred embodiments described herein. Upon reading this specification, those skilled in the art will recognize various modifications thereof. Therefore, it is to be understood that such modifications are intended to fall within the scope of the appended claims.

All references cited in the present application are incorporated by reference herein to the extent that there is no inconsistency with the present disclosure.

### References

1. (1998). Genome sequence of the nematode C. elegans: a platform for investigating biology. The C. elegans Sequencing Consortium. Science 282, 2012-8.

2. Adams, M. D., Celniker, S. E., Holt, R. A., Evans, C. A., Gocayne, J. D., Amanatides, P. G., Scherer, S. E., Li, P. W., Hoskins, R. A., Galle, R. F., George, R. A., Lewis, S. E., Richards, S., Ashburner, M., Henderson, S. N., Sutton, G. G., Wortman, J. R., Yandell, M. D., Zhang, Q., Chen, L. X., Brandon, R. C., Rogers, Y. H., Blazej, R. G., Champe, M., Pfeiffer, B. D., Wan, K. H., Doyle, C., Baxter, E. G., Helt, G., Nelson, C. R., Gabor, G. L., Abril, J. F., Agbayani, A., An, H. J., Andrews-Pfannkoch, C., Baldwin, D., Ballew, R. M., Basu, A., Baxendale, J., Bayraktaroglu, L., Beasley, E. M., Beeson, K. Y., Benos, P. V., Berman, B. P., Bhandari, D., Bolshakov, S., Borkova, D., Botchan, M. R., Bouck, J., Brokstein, P., Brottier, P., Burtis, K. C., Busam, D. A., Butler, H., Cadieu, E., Center, A., Chandra, I., Cherry, J. M., Cawley, S., Dahike, C., Davenport, L. B., Davies, P., de Pablos, B., Delcher, A., Deng, Z., Mays, A. D., Dew, I., Dietz, S. M., Dodson, K., Doup, L. E., Downes, M., Dugan-Rocha, S., Dunkov, B. C., Dunn, P., Durbin, K. J., Evangelista, C. C., Ferraz, C., Ferriera, S., Fleischmann, W., Fosler, C., Gabrielian, A. E., Garg, N. S., Gelbart, W. M., Glasser, K., Glodek, A., Gong, F., Gorrell, J. H., Gu, Z., Guan, P., Harris, M., Harris, N. L., Harvey, D., Heiman, T. J., Hernandez, J. R., Houck, J., Hostin, D., Houston, K. A., Howland, T. J., Wei, M. H., Ibegwam, C., et al. (2000). The genome sequence of Drosophila melanogaster. Science 287, 2185-95.Bechtold, N., Ellis, J., and Pelletier, G. (1993). In planta Agrobacterium mediated gene transfer by infiltration of adult Arabidopsis thaliana plants. C.R.Acad.Sci.Paris, Sciences de la vie 316, 1194-1199.

3. Allenbach, L., and Poirier, Y. (2000). Analysis of the alternative pathways for the beta-oxidation of unsaturated fatty acids using transgenic plants synthesizing polyhydroxyalkanoates in peroxisomes. Plant Physiol 124, 1159-68.

4. Anderson, A. J., and Dawes, E. A. (1990). Occurrence, metabolism, metabolic role, and industrial uses of bacterial polyhydroxyalkanoates. Microbiol Rev 54, 450-72.

5. Becker, A., Hessenius, C., Licha, K., Ebert, B., Sukowski, U., Semmler, W., Wiedenmann, B., and Grotzinger, C. (2001). Receptor-targeted optical imaging of tumors with near-infrared fluorescent ligands. Nat Biotechnol 19, 327-31.

6. Beerli, R. R., Dreier, B., and Barbas, C. F., 3rd (2000). Positive and negative regulation of endogenous genes by designed transcription factors. Proc Natl Acad Sci USA 97, 1495-500.

7. Beerli, R. R., Schopfer, U., Dreier, B., and Barbas, C. F., 3rd (2000). Chemically regulated zinc finger transcription factors. J Biol Chem 275, 32617-27.

8. Bent, A. F. (2000). Arabidopsis in planta transformation. Uses, mechanisms, and prospects for transformation of other species. Plant Physiol 124, 1540-7.

9. Beveridge, J. A., Riseman, E. M., and Graves, C. R. (1997). How easy is matching 2D line models using local search? IEEE Trans. Pattern Analysis and Machine Intelligence 19, 564-579.

10. Beveridge, J. R., She, K., Draper, B., and Givens, G. H. (2001). A nonparametric statistical comparison of principal component and linear discriminat subspaces for face recognition. Proceedings of the IEEE Conference on Pattern Recognition and Machine Intelligence in press.Billinton, N., and Knight, A. W. (2001). Seeing the Wood through the Trees: A Review of Techniques for Distinguishing Green Fluorescent Protein from Endogenous Autofluorescence. Anal Biochem 291, 175-97.

11. Bohmert, K., Balbo, I., Kopka, J., Mittendorf, V., Nawrath, C., Poirier, Y., Tischendorf, G., Trethewey, R. N., and Willmitzer, L. (2000). Transgenic Arabidopsis plants can accumulate polyhydroxybutyrate to up to 4% of their fresh weight. Planta 211, 841-5.

12. Bohmert, K., Balbo, I., Kopka, J., Mittendorf, V., Nawrath, C., Poirier, Y., Tischendorf, G., Trethewey, R. N., and Willmitzer, L. (2000). Transgenic Arabidopsis plants can accumulate polyhydroxybutyrate to up to 4% of their fresh weight.[In Process Citation]. Planta211, 841-5.

13. Boppart, S., Brezinski, M., Bouma, B., Tearney, G., and Fujimoto, J. (1997). Investigation of Developing Embryonic Morphology Using Optical Coherence Tomography. Developmental Biology 177, 54-63.

14. Boppart, S., Tearney, G., Bouma, B., Southern, J., Brezinski, M., and Fujimoto, J. (1997). Noninvasive assessment of the developing Xenopus cardiovascular system using optical coherence tomography. Proc.Natl.Acad.Sci.USA 94, 4256-4261.

15. Boppart, S. A., Bouma, B. E., Pitris, C., Southern, J. F., Brezinski, M. E., and Fujimoto, J. G. (1998). In vivo cellular optical coherence tomography imaging. Nat Med 4, 861-5.

16. Boppart, S. A., Bouma, B. E., Pitris, C., Tearney, G. J., Southern, J. F., Brezinski, M. E., and Fujimoto, J. G. (1998). lntraoperative assessment of microsurgery with three-dimensional optical coherence tomography. Radiology 208, 81-86.

17. Boppart, S. A., Brezinski, M. E., Pitris, C., and Fujimoto, J. G. (1998). Optical coherence tomography for neurosurgical imaging of human intracortical melanoma. Neurosurgery 43, 834-841.

18. Boppart, S. A., Herrmann, J., Pitris, C., Stamper, D. L., Brezinski, M. E., and Fujimoto, J. G. (1999). High-Resolution Optical Coherence Tomography-Guided Laser Ablation of Surgical Tissue. J Surg Res 82, 275-284.

19. Boppart, S. A., Brezinski, M. E., and Fujimoto, J. G. (2000). Optical coherence tomography imaging in developmental biology. Methods Mol Biol 135, 217-33.

20. Blake, A., and Isard, M. (1999). Active Contours: The Application of Techniques from Graphics, Vision, Control Theory and Statistics to Visual Tracking of Shapes in Motion (New York: Springer).

21. Blumenthal, E. Z., Williams, J. M., Weinreb, R. N., Girkin, C. A., Berry, C. C., and Zangwill, L. M. (2000). Reproducibility of nerve fiber layer thickness measurements by use of optical coherence tomography. Ophthalmology 107, 2278-82.Boppart, S., Tearney, G., Bouma, B., Southern, J., Brezinski, M., and Fujimoto, J. (1997). Noninvasive assessment of the developing Xenopus cardiovascular system using optical coherence tomography. Proc.Natl.Acad.Sci.USA 94, 4256-4261.

22. Bohmert, K., Balbo, I., Kopka, J., Mittendorf, V., Nawrath, C., Poirier, Y., Tischendorf, G., Trethewey, R. N., and Willmitzer, L. (2000). Transgenic Arabidopsis plants can accumulate polyhydroxybutyrate to up to 4% of their fresh weight. Planta 211, 841-5.

23. Boppart, S. A., Bouma, B. E., Pitris, C., Southern, J. F., Brezinaki, M. E., and Fujimoto, J. G. (1998). In vivo cellular optical coherence tomography imaging. Nat Med 4, 861-5.

24. Boppart, S. A., Bouma, B. E., Pitris, C., Tearney, G. J., Southern, J. F., Brezinski, M. E., and Fujimoto, J. G. (1998). Intraoperative assessment of microsurgery with three-dimensional optical coherence tomography. Radiology 208, 81-86.

25. Boppart, S. A., Brezinski, M. E., Pitris, C., and Fujimoto, J. G. (1998). Optical coherence tomography for neurosurgical imaging of human intracortical melanoma. Neurosurgery 43, 834-841.

26. Boppart, S. A., Herrmann, J., Pitris, C., Stamper, D. L., Brezinski, M. E., and Fujimoto, J. G. (1999). High-Resolution Optical Coherence Tomography-Guided Laser Ablation of Surgical Tissue. J Surg Res 82, 275-284.

27. Boyes, D. C., Zayed, A. M., Ascenzi, R., McCaskill, A. J., Hoffman, N. E., Davis, K. R., and Gorlach, J. (2001). Growth stage-based phenotypic analysis of arabidopsis: a model for high throughput functional genomics in plants. Plant Cell 13, 1499-510.

28. Capecchi (1989), Science 244, 1288-1292.

29. Chauhan, D. S., and Marshall, J. (1999). The interpretation of optical coherence tomography images of the retina. Invest Ophthalmol Vis Sci 40, 2332-42.

30. Christensen, H. E., Ramachandran, S., Tan, C. T., Surana, U., Dong, C. H., and Chua, N. H. (1996). Arabidopsis profilins are functionally similar to yeast profilins: identification of a vascular bundle-specific profilin and a pollen-specific profilin. Plant J 10, 269-79.

31. Clark, S. E., Jacobsen, S. E., Levin, J. Z., and Meyerowitz, E. M. (1996). The CLAVATA and SHOOT MERISTEMLESS loci competitively regulate meristem activity in Arabidopsis. Development 122, 1567-75.

32. Clark, S. E. (1997) Organ formation at the vegetative shoot apical meristem. The Plant Cell 9: 1067-1076.

33. Colon-Carmona, A., You, R., Haimovitch-Gal, T., and Doerner, P. (1999). Technical advance: spatio-temporal analysis of mitotic activity with a labile cyclin-GUS fusion protein. Plant J 20, 503-8.

34. Contag, C. H., Fraser, S., and Weissleder, R. (2000). Strategies in in vivo molecular imaging. NeoReviews 1, e225-e232.

35. Contag, C. H., Weissleder, R., Bachmann, M. H., and Fraser, S. E. (2000). Applications of in vivo molecular imaging in biology and medicine. NeoReviews 1, e233-e240.

36. Cutler, S. R., Ehrhardt, D. W., Griffitts, J. S., and Somerville, C. R. (2000). Random GFP:cDNA fusions enable.visualization of subcellular structures in cells of Arabidopsis at a high frequency. Proc Natl Acad Sci USA 97, 3718-23.

37. D'Amico, A. V., Weinstein, M., Li, X., Richie, J. P., and Fujimoto, J. (2000). Optical coherence tomography as a method for identifying benign and malignant microscopic structures in the prostate gland. Urology 55, 783-7.

38. Davenport, R. J. (2001). Rice genome. Syngenta finishes, consortium goes on. Science 291, 807.

39. Dreier, B., Beerli, R. R., Segal, D. J., Flippin, J. D., and Barbas, I. (2001). Development of zinc finger domains for recognition of the 5′-ANN-3′ family of DNA sequences and their use in the construction of artificial transcription factors. J Biol Chem 4, 4.

40. Drexler, W., Morgner, U., Ghanta, R. K., Kartner, F. X., Schuman, J. S., and Fujimoto, J. G. (2001). Ultrahigh-resolution ophthalmic optical coherence tomography. Nat Med 7, 502-7.

41. Drezek, R., Dunn, A., and Richards-Kortum, R. (1999). Light scattering from cells: finite-difference time-domain simulations and goniometric measurements. Applied optics 38, 3651-3661.

42. Drummond, T., and Cipolla, R. (1999). Real-time tracking of complex structures with on-line camera calibration. Proceedings of British Machine Vision Conference 1999, 574-583

43. Dunn, A., and Richards-Kotum, R. (1996). Three-dimensional computation of light scattering from cells. IEEE J. of Selected Topics in Quantum Electronics 2, 898-905.

44. Faust, M., Wang, P. C., and Maas, J. (1997). The use of Magnetic Resonance Imaging in Plant Science. Horticultural Reviews 20, 225-266.

45. Fercher, A. F. (1996). Optical coherence tomography. J Biomed Optics 1, 157-173.

46. Fletcher, J. C., Brand, U., Running, M. P., Simon, R., and Meyerowitz, E. M. (1999). Signaling of cell fate decisions by CLAVATA3 in Arabidopsis shoot meristems. Science 283, 1911-4.

47. Forbes, L. A. (2001). Object recognition using an extended condensation filter. Ph.D. Dissertation in Computer Science. Fort Collins, Colorado State University.

48. Fujimoto, J. G., Bouma, B., Tearney, G. J., Boppart, S. A., Pitris, C., Southern, J. F., and Brezinski, M. E. (1998). New technology for high-speed and high-resolution optical coherence tomography. Ann N Y Acad Sci 838, 95-107.

49. Fujimoto, J. G., Pitris, C., Boppart, S. A., and Brezinski, M. E. (2000). Optical coherence tomography: an emerging technology for biomedical imaging and optical biopsy. Neoplasia 2, 9-25.

50. Gambhir, S. S., Barrio, J. R., Phelps, M. E., lyer, M., Namavari, M., Satyamurthy, N., Wu, L., Green, L. A., Bauer, E., MacLaren, D. C., Nguyen, K., Berk, A. J., Cherry, S. R., and Herschman, H. R. (1999). Imaging adenoviral-directed reporter gene expression in living animals with positron emission tomography. Proc Natl Acad Sci USA 96, 2333-8.

51. Gambhir, S. S., Bauer, E., Black, M. E., Liang, Q., Kokoris, M. S., Barrio, J. R., lyer, M., Namavari, M., Phelps, M. E., and Herschman, H. R. (2000). A mutant herpes simplex virus type 1 thymidine kinase reporter gene shows improved sensitivity for imaging reporter gene expression with positron emission tomography. Proc Natl Acad Sci USA 97, 2785-90.

52. Gao, D., Maehara, A., Yamane, T., and Ueda, S. (2001). Identification of the intracellular polyhydroxyalkanoate depolymerase gene of Paracoccus denitrificans and some properties of the gene product. FEMS Microbiol Lett 196, 159-64.

53. Gray, J. D., Kolesik, P., Hoj, P. B., and Coombe, B. G. (1999). Confocal measurement of the three-dimensional size and shape of plant parenchyma cells in developing fruit tissue. The Plant Journal 19, 229-236.

54. Grimson, W. E. L. (1990). Object Recognition by Computer: The Role of Geometric Constraints (Boston: MIT Press).

55. Gura, T. (1997). Biologists get up close and personal with live cells. Science 276, 1988-1990.

56. Gurses-Ozden, R., Ishikawa, H., Hoh, S. T., Liebmann, J. M., Mistiberger, A., Greenfield, D. S., Dou, H. L., and Ritch, R. (1999). Increasing sampling density improves reproducibility of optical coherence tomography measurements. J Glaucoma 8, 238-41.

57. Handrick, R., Reinhardt, S., and Jendrossek, D. (2000). Mobilization of poly(3-hydroxybutyrate) in Ralstonia eutropha. J Bacteriol 182, 5916-8.

58. Hanson, M. R., and Kohler, R. H. (2001). GFP imaging: methodology and application to investigate cellular compartmentation in plants. J. Exp. Botany 52, 529-539.

59. Haseloff, J. (1999). GFP variants for multispectral imaging of living cells. Methods in Cell Biology 58, 139-151.

60. Haseloff, J., Dormand, E.-L., and Brand, A. H. (1999). Live imaging of gene fluorescent protein. In Methods in Molecular Biology: Protocols in Confocal Microscopy, S. Paddock, ed. (Totowa, N.J.: Humana Press).

61. Herrmann, J. M., Brezinski, M. E., Bouma, B. E., Boppart, S. A., Pitris, C., Southern, J. F., and Fujimoto, J. G. (1998). Two- and three-dimensional high-resolution imaging of the human oviduct with optical coherence tomography. Fertil Steril 70, 155-8.

62. Herschman, H. R., MacLaren, D. C., Iyer, M., Namavari, M., Bobinski, K., Green, L. A., Wu, L., Berk, A. J., Toyokuni, T., Barrio, J. R., Cherry, S. R., Phelps, M. E., Sandgren, E. P., and Gambhir, S. S. (2000). Seeing is believing: non-invasive, quantitative and repetitive imaging of reporter gene expression in living animals, using positron emission tomography. J Neurosci Res 59, 699-705.

63. Hettinger, J. W., de la Pena Mattozzi, M., Meyers, W., Williams, M. E., Reeves, A., Parsons, R. L., Haskell, R. C., Petersen, D. C., Wang, R., and Medford, J. I. (2000). Optical Coherence Microscopy: a technology for rapid, in vivo, non-destructive visualization of plants and plant cells. Plant Physiology 123, 3-15.

64. Hettinger, J. W. (2001). Enhancement of an in vivo imaging technology for plants, Optical Coherence Microscopy. Masters Thesis. Fort Collins: Colorado State University, pp. 216.

65. Hirakawa, H., Iijima, H., Gohdo, T., and Tsukahara, S. (1999). Optical coherence tomography of cystoid macular edema associated with retinitis pigmentosa. Am J Ophthalmol 128, 185-91.

66. Histand, M., and Alciatore, D. (1999). Introduction to mechantronics and measurement systems, 1st Edition (New York: McGraw-Hill).

67. Hoeling, B. M., Fernandez, A. D., Haskell, R. C., Myers, W. R., Petersen, D. C., Ungersma, S. E., Wang, R., Williams, M. E., and Fraser, S. E. (2000). An optical coherence microscope for 3-Dimensional imaging in developmental biology. Optics Express 6:136-146.

68. Hoeling, B. M., Fernandez, A. D., Haskell, R. C., and Petersen, D. C. (2000). Phase modulation at 100 kHZ in a Michelson Interferometer using an inexpensive piezoelectric stack driven at resonance. Review of Scientific Instruments 72, 1630-1633.

69. Hogan et al., “Manipulating the Mouse Embryo, A Laboratory Manual,” Cold Spring Harbor Laboratory.

70. Holbrook, N. M., Ahrens, E. T., Burns, M. J., and Zwieniecki, M. A. (2001). In vivo observation of cavitation and embolism repair using magnetic resonance imaging. Plant Physiol 126, 27-31.

71. Huang, D., Swanson, E. A., Lin, C. P., Schuman, J. S., Stinson, W. G., Chang, W., Hee, M. R., Flofte, T., Gregory, K., Puliafito, C. A., and et al. (1991). Optical coherence tomography. Science 254, 1178-81.

72. Huttenlocher, D. P., Klanderman, G. A., and Rucklidge, W. J. (1993). Comparing imanges using the Hausdorff. IEEE Trans. Pattern Analysis and Machine Intelligence 15, 850-862.

73. Igarashi, M., Demura, T., and Fukuda, H. (1998). Expression of the Zinnia TED3 promoter in developing tracheary elements of transgenic Arabidopsis. Plant Mol Biol 36, 917-27.

74. Initiative, The Arabidosis Genome Initiative (2000). Analysis of the genome sequence of the flowering plant Arabidopsis thaliana. Nature 408, 796-815.

75. Izatt, J. A., Kulkarni, M. D., Wang, H. -W., Kobayashi, K., and Sivak, M. V. J. (1996). Optical coherence tomography and microscopy in gastrointestinal tissues. IEEE J Sel Topics Quantum Electron 2, 1017-1028.

76. Jefferson, R. A., Kavanagh, T. A., and Bevan, M. W. (1987). GUS fusions: B-glucuronidase as a sensitive and versatile gene fusion marker in higher plants. EMBO J. 6, 3901-3907.

77. Kessler, B., Weusthuis, R., Witholt, B., and Eggink, G. (2001). Production of microbial polyesters: fermentation and downstream processes. Adv Biochem Eng Biotechnol 71, 159-82.

78. Klinke, S., de Roo, G., Witholt, B., and Kessler, B. (2000). Role of phaD in accumulation of medium-chain-length Poly (3-hydroxyalkanoates) in Pseudomonas oleovorans. Appl Environ Microbiol 66, 3705-10.

79. Kockenberger, W. (2001). Functional imaging of plants by magnetic resonance experiments. Trends Plant Sci 6, 286-92.

80. Kockenberger, W. (2001). Nuclear magnetic resonance micro-imaging in the investigation of plant cell metabolism. J Exp Bot 52, 641-52.

81. Kubigsteltig, I., Laudert, D., and Weiler, E. W. (1999). Structure and regulation of the Arabidopsis thaliana allene oxide synthase gene. Planta 208, 463-71.

82. Kuchenbrod, E., Kahler, E., Thurmer, F., Deichmann, R., Zimmermann, U., and Haase, A. (1998). Functional magnetic resonance imaging in intact plants—quantitative observation of flow in plant vessels. Magnetic Resonance Imaging 16, 331-338.

83. Laufs, P., Grandjean, O., Jonak, C., and Traas, J. (1998). Cellular parameters of the shoot apical meristem in Arabidopsis. The Plant Cell 10, 1375-1389.

84. Leaf, T. A., Peterson, M. S., Stoup, S. K., Somers, D., and Srienc, F. (1996). Saccharomyces cerevisiae expressing bacterial polyhydroxybutyrate synthase produces poly-3-hydroxybutyrate. Microbiology 142, 1169-80.

85. Liu, H. S., Jan, M. S., Chou, C. K., Chen, P. H., and Ke, N. J. (1999). Is green fluorescent protein toxic to the living cells? Biochem Biophys Res Commun 260, 712-7.

86. Lok, C. (2001). Picture perfect. Nature 412, 3724.

87. Louie, A. Y., Huber, M. M., Ahrens, E. T., Rothbacher, U., Moats, R., Jacobs, R. E., Fraser, S. E., and Meade, T. J. (2000). In vivo visualization of gene expression using magnetic resonance imaging. Nat Biotechnol 18, 321-5.

88. Lowe, D. G. (1985). Perceptual Organization and Visual Recognition (Dordrecht: Kluwer).

89. Lynn, K., Fernandez, A., Aida, M., Sedbrook, J., Tasaka, M., Masson, P., and Barton, M. K. (1999). The PINHEAD/ZWILLE gene acts pleiotropically in Arabidopsis development and has overlapping functions with the ARGONAUTE1 gene. Development 126, 469-81.

90. Martienssen, R. A. (1998). Functional genomics: probing plant gene function and expression with transposons. Proc Natl Acad Sci USA95, 2021-6.

91. Masters, B. R. (1999). Early development of optical low-coherence reflectometry and some recent biomedical applications. J Biomed Optics 4, 236-247.

92. Medford, J. I. (1992). Vegetative apical meristems. The Plant Cell 4, 1029-1039.

93. Medford, J. I., Behringer, F. J., Callos, J. D., and Feldmann, K. A. (1992). Normal and abnormal development in the Arabidopsis vegetative shoot apex. The Plant Cell 4, 631-643.

94. Medford, J. I., Link, B. M., and Callos, J. D. (1994). Interaction and function of the Arabidopsis Forever young gene in the shoot apical meristem.

95. Medford, J. I., Haskell, R. C., Hoeling, B. M., Petersen, D. C., Wang, R., and Williams, M. (2001). Optical coherence microscope and methods of use for rapid in vivo three-dimensional visualization of biological function: Harvey Mudd College).

96. Medford, J. I., Parsons, R. L., Reeves, A., and Hettinger, J. W. (2001). In vivo gene expression Patent Pending. No. 60/117,963

97. Meier, C., Bouquin, T., Nielsen, M. E., Raventos, D., Mattsson, O., Rocher, A., Schomburg, F., Amasino, R. M., and Mundy, J. (2001). Gibberellin response mutants identified by luciferase imaging. Plant J 25, 509-19.

98. Mewes, H. W., Albermann, K., Bahr, M., Frishman, D., Gleissner, A., Hani, J., Heumann, K., Kleine, K., Maierl, A., Oliver, S. G., Pfeiffer, F., and Zollner, A. (1997). Overview of the yeast genome. Nature 387, 7-65.

99. Meyerowitz, E. M. (2001). Prehistory and history of arabidopsis research. Plant Physiol 125, 15-9.

100. Millar, A. J., Short, S. R., Chua, N. H., and Kay, S. A. (1992). A novel circadian phenotype based on firefly luciferase expression in transgenic plants. Plant Cell 4, 1075-87.

101. Mistiberger, A., Liebmann, J. M., Greenfield, D. S., Pons, M. E., Hoh, S. T., Ishikawa, H., and Ritch, R. (1999). Heidelberg retina tomography and optical coherence tomography in normal, ocular-hypertensive, and glaucomatous eyes. Ophthalmology 106, 2027-32.

102. Nawrath, C., Poirier, Y., and Somerville, C. (1994). Targeting of the polyhydroxybutyrate biosynthetic pathway to the plastids of Arabidopsis thalaina results in high levels of polymer accumulation. Proceedings of the National Academy of Sciences USA 26, 12760-12764.

103. Ohura, T., Kasuya, K. I., and Doi, Y. (1999). Cloning and characterization of the polyhydroxybutyrate depolymerase gene of Pseudomonas stutzeri and analysis of the function of substrate-binding domains. Appl Environ Microbiol 65, 189-97.

104. Oppenheimer, D. G., Herman, P. L., Sivakumaran, S., Esch, J., and Marks, M. D. (1991). A myb gene required for leaf trichome differentiation in Arabidopsisis expressed in stipules. Cell 67, 483-493.

105. Ostle, A. G., and Holt, J. G. (1982). Nile blue A as a fluorescent stain for poly-beta-hydroxybutyrate. Appl Environ Microbiol 44, 238-41.

106. Parisi, V., Manni, G., Spadaro, M., Colacino, G., Restuccia, R., Marchi, S., Bucci, M. G., and Pierelli, F. (1999). Correlation between morphological and functional retinal impairment in multiple sclerosis patients. Invest Ophthalmol Vis Sci 40, 2520-7.

107. Phelps, M. E. (2000). Inaugural article: positron emission tomography provides molecular imaging of biological processes. Proc Natl Acad Sci USA 97, 9226-33.

108. Piston, D. W. (1999). Imaging living cells and tissues by two-photon excitation microscopy. Trends Cell Biol 9, 66-9.

109. Pitris, C., Saunders, K. T., Fujimoto, J. G., and Brezinski, M. E. (2001). High-resolution imaging of the middle ear with optical coherence tomography: a feasibility study. Arch Otolaryngol Head Neck Surg 127, 637-42.

110. Poethig, R. S. (1997). Leaf morphogenesis in flowering plants. The Plant Cell 9, 1077-087.

111. Poirier, Y., Dennis, D. E., Klomparens, K., and Somerville, C. (1992). Polyhydroxybutyrate, a biodegradable thermoplastic, produced in transgenic plants. Science 256, 520-523.

112. Poirier, Y., Dennis, D. E., Nawrath, C., and Somerville, C. (1993). Progress Toward Biologically Produced Biodegradable Thermoplastics. Adv Mater 5, 30-37.

113. Poirier, Y., Nawrath, C., and Somerville, C. (1995). Production of polyhydroxyalkanoates, a family of biodegradable plastics and elastomers, in bacteria and plants. Biotechnology (N Y) 13, 142-50.

114. Poirier, Y., Somerville, C., Schechtman, L. A., Satkowski, M. M., and Noda, I. (1995). Synthesis of high-molecular-weight poly([R]-(−)-3-hydroxybutyrate) in transgenic Arabidopsis thaliana plant cells. Int J Biol Macromol 17, 7-12.

115. Poirier, Y. (1999). Production of new polymeric compounds in plants. Curr Opin Biotechnol 10, 181-5.

116. Poirier, Y., Ventre, G., and Caldelari, D. (1999). Increased flow of fatty acids toward beta-oxidation in developing seeds of Arabidopsis deficient in diacylglycerol acyltransferase activity or synthesizing medium-chain-length fatty acids. Plant Physiol 121, 1359-66.

117. Poirier, Y. (2001). Production of polyesters in transgenic plants. Adv Biochem Eng Biotechnol 71, 209-40.

118. Radhakrishnan, S., Rollins, A. M., Roth, J. E., Yazdanfar, S., Westphal, V., Bardenstein, D. S., and Izatt, J. A. (2001). Real-time optical coherence tomography of the anterior segment at 1310 nm. Arch Ophthalmol 119, 1179-85.

119. Ratcliffe, R. G., and Shachar-Hill, Y. (2001). Probing plant metabolism with NMR. Annu Rev Plant Physiol Plant Mol Biol 52, 499-526.

120. Reeves, A., R. L. Parsons and J. I. Medford. (2001) A rapid, three-dimensional technique for in vivo analysis of mutations and responses in plants (submitted).

121. Ripandelli, G., Coppe, A. M., Capaldo, A., and Stirpe, M. (1998). Optical Coherence Tomography. Seminar in Ophthalmology 13, 199-202.

122. Running, M. P., Clark, S. E., and Meyerowitz, E. M. (1995). Confocal microscopy of the shoot apex. Methods in Cell Biology 49, 217-229.

123. Saegusa, H., Shiraki, M., Kanai, C., and Saito, T. (2001). Cloning of an intracellular Poly[D(−)-3-Hydroxybutyrate] depolymerase gene from Ralstonia eutropha H16 and characterization of the gene product. J Bacteriol 183, 94-100.

124. Schober, U., Thiel, C., and Jendrossek, D. (2000). Poly(3-hydroxyvalerate) depolymerase of Pseudomonas lemoignei. Appl Environ Microbiol 66, 1385-92.

125. Segal, D. J., Dreier, B., Beerli, R. R., and Barbas, C. F., 3rd (1999). Toward controlling gene expression at will: selection and design of zinc finger domains recognizing each of the 5′-GNN-3′ DNA target sequences. Proc Natl Acad Sci USA 96, 2758-63.

126. Shi, N., Boado, R. J., and Pardridge, W. M. (2000). Antisense imaging of gene expression in the brain in vivo. Proc Natl Acad Sci USA 97, 14709-14714.

127. Siegfried, K. R., Eshed, Y., Baum, S. F., Otsuga, D., Drews, G. N., and Bowman, J. L. (1999). Members of the YABBY gene family specify abaxial cell fate in Arabidopsis. Development 126, 4117-28.

128. Slater, S., Mitsky, T. A., Houmiel, K. L., Hao, M., Reiser, S. E., Taylor, N. B., Tran, M., Valentin, H. E., Rodriguez, D. J., Stone, D. A., Padgette, S. R., Kishore, G., and Gruys, K. J. (1999). Metabolic engineering of Arabidopsis and Brassica for poly(3-hydroxybutyrate-co-3-hydroxyvalerate) copolymer production. Nat Biotechnol 17, 1011-6.

129. Sokolov, K., Drezek, R., Gossage, K., and Richards-Kortum, R. (1999). Reflectrance spectroscopy with polarized light: is it sensitive to cellular and nuclear morphology. Optics Express 5, 302-317.

130. Somerville, C., and Somerville, S. (1999). Plant Functional Genomics. Science 285, 380-383.

131. Springer, P. S. (2000). Gene traps: tools for plant development and genomics. Plant Cell 12, 1007-20.

132. Steeves, T. A., and Sussex, I. M. (1989). Patterns in plant development, 2 Edition (New York: Cambridge University Press).

133. Stevens, M. R., and Beveridge, J. R. (2000). Integrating graphics and vision for object recognition (Dordrecht: Kluwer Academic Publishers).

134. Swanson, E. A., Izatt, J. A., Hee, M. R., Huang, D., Lin, C. P., Schuman, J. S., Puliafito, C. A., and Fujimoto, J. G. (1993). In vivo retinal imaging by optical coherence tomography. Optics Letters 18, 1864-1869.

135. Tadrous, P. J. (2000). Methods for imaging the structure and function of living tissues and cells: 1. Optical coherence tomography. J Pathol 191, 115-9.

136. Taubes, G. (1997). Play of light opens a new window into the body. Science 276, 1991-1993.

137. Tearney, G. J., Brezinski, M. E., Bouma, B. E., Boppart, S. A., Pitris, C., Southern, J. F., and Fujimoto, J. G. (1997). In vivo endoscopic optiocal biopsy with optical coherence tomography. Science 276, 2037-2039.

138. Tepler, A., and Poethig, R. S. (1994). Leaf development in Arabidopsis. In ARABIDOPSIS, E. M. Meyerowitz and C. R. Somerville, eds. (Cold Spring Harbor: Cold Spring Harbor Press), pp. 379-401.

139. Thain, S. C., Hall, A., and Millar, A. J. (2000). Functional independence of circadian clocks that regulate plant gene expression. Curr Biol 10, 951-6.

140. Timmermans, M. C. P., Schultes, N. P., Jankovsky, J. P., and Nelson, T. (1998). Leafbladeless1 is required for dorsoventrality of lateral organs in maize. Development 125, 2813-2823.

141. Torii, K. U., Mitsukawa, N., Oosumi, T., Matsuura, Y., Yokoyama, R., Whittier, R. F., and Komeda, Y. (1996). The Arabidopsis ERECTA gene encodes a putative receptor protein kinase with extracellular leucine-rich repeats. Plant Cell 8, 735-46.

142. Trotochaud, A. E., Hao, T., Wu, G., Yang, Z., and Clark, S. E. (1999). The CLAVATAL receptor-like kinase requires CLAVATA3 for its assembly into a signaling complex that includes KAPP and a rho-related protein. Plant Cell 11, 393-406.

143. Trotochaud, A. E., Jeong, S., and Clark, S. E. (2000). CLAVATA3, a multimeric ligand for the CLAVATA1 receptor-kinase. Science 289, 613-7.

144. Turk, M. A., and Pentland, A. P. (1991). Face recognition using Eigenfaces. Proc. IEEE Conference on Computer Vision and Pattern Recognition, 586-591.

145. van der Walle, G. A. M., de Konning, G. J. M., Weusthuis, R. A., and Eggink, G. (2001). Properties, modifications and applications of biopolyesters. Adv Biochem Eng Biotechnol 71, 264-289.

146. Venter, J. C., Adams, M. D., Myers, E. W., Li, P. W., Mural, R. J., Sutton, G. G., Smith, H. O., Yandell, M., Evans, C. A., Holt, R. A., Gocayne, J. D., Amanatides, P., Ballew, R. M., Huson, D. H., Wortman, J. R., Zhang, Q., Kodira, C. D., Zheng, X. H., Chen, L., Skupski, M., Subramanian, G., Thomas, P. D., Zhang, J., Gabor Miklos, G. L., Nelson, C., Broder, S., Clark, A. G., Nadeau, J., McKusick, V. A., Zinder, N., Levine, A. J., Roberts, R. J., Simon, M., Slayman, C., Hunkapiller, M., Bolanos, R., Delcher, A., Dew, I., Fasulo, D., Flanigan, M., Florea, L., Halpern, A., Hannenhalli, S., Kravitz, S., Levy, S., Mobarry, C., Reinert, K., Remington, K., Abu-Threideh, J., Beasley, E., Biddick, K., Bonazzi, V., Brandon, R., Cargill, M., Chandramouliswaran, I., Charlab, R., Chaturvedi, K., Deng, Z., Di Francesco, V., Dunn, P., Eilbeck, K., Evangelista, C., Gabrielian, A. E., Gan, W., Ge, W., Gong, F., Gu, Z., Guan, P., Heiman, T. J., Higgins, M. E., Ji, R. R., Ke, Z., Ketchum, K. A., Lai, Z., Lei, Y., Li, Z., Li, J., Liang, Y., Lin, X., Lu, F., Merkulov, G. V., Milshina, N., Moore, H. M., Naik, A. K., Narayan, V. A., Neelam, B., Nusskern, D., Rusch, D. B., Salzberg, S., Shao, W., Shue, B., Sun, J., Wang, Z., Wang, A., Wang, X., Wang, J., Wei, M., Wides, R., Xiao, C., Yan, C., et al. (2001). The sequence of the human genome. Science 291, 1304-51.

147. 26. Venter, J. C., Adams, M. D., Myers, E. W., Li, P. W., Mural, R. J., Sutton, G. G., Smith, H. O., Yandell, M., Evans, C. A., Holt, R. A., Gocayne, J. D., Amanatides, P., Ballew, R. M., Husbn, D. H., Wortman, J. R., Zhang, Q., Kodira, C. D., Zheng, X. H., Chen, L., Skupski, M., Subramanian, G., Thomas, P. D., Zhang, J., Gabor Miklos, G. L., Nelson, C., Broder, S., Clark, A. G., Nadeau, J., McKusick, V. A., Zinder, N., Levine, A. J., Roberts, R. J., Simon, M., Slayman, C., Hunkapiller, M., Bolanos, R., Delcher, A., Dew, I., Fasulo, D., Flanigan, M., Florea, L., Halpern, A., Hannenhalli, S., Kravitz, S., Levy, S., Mobarry, C., Reinert, K., Remington, K., Abu-Threideh, J., Beasley, E., Biddick, K., Bonazzi, V., Brandon, R., Cargill, M., Chandramouliswaran, I., Charlab, R., Chaturvedi, K., Deng, Z., Di Francesco, V., Dunn, P., Eilbeck, K., Evangelista, C., Gabrielian, A. E., Gan, W., Ge, W., Gong, F., Gu, Z., Guan, P., Heiman, T. J., Higgins, M. E., Ji, R. R., Ke, Z., Ketchum, K. A., Lai, Z., Lei, Y., Li, Z., Li, J., Liang, Y., Lin, X., Lu, F., Merkulov, G. V., Milshina, N., Moore, H. M., Naik, A. K., Narayan, V. A., Neelam, B., Nusskern, D., Rusch, D. B., Salzberg, S., Shao, W., Shue, B., Sun, J., Wang, Z., Wang, A., Wang, X., Wang, J., Wei, M., Wides, R., Xiao, C., Yan, C., et al. (2001). The sequence of the human genome. Science 291, 1304-51.

148. Weissleder, R., Moore, A., Mahmood, U., Bhorade, R., Benveniste, H., Chiocca, E. A., and Basilion, J. P. (2000). In vivo magnetic resonance imaging of transgene expression. Nat Med 6, 351-5.

149. Williams, M. D., Rahn, J. A., and Sherman, D. H. (1996). Production of a polyhydroxyalkanoate biopolymer in insect cells with a modified eucaryotic fatty acid synthase. Appl Environ Microbiol 62, 2540-6.

150. Worley, C. K., Ling, R., and Callis, J. (1998). Engineering in vivo instability of firefly luciferase and Escherichia coli beta-glucuronidase in higher plants using recognition elements from the ubiquitin pathway. Plant Mol Biol 37, 337-47.

151. Yang, M., Baranov, E., Jiang, P., Sun, F. X., Li, X. M., Li, L., Hasegawa, S., Bouvet, M., Al-Tuwaijri, M., Chishima, T., Shimada, H., Moossa, A. R., Penman, S., and Hoffman, R. M. (2000). Whole-body optical imaging of green fluorescent protein-expressing tumors and metastases. Proc Natl Acad Sci USA 97, 1206-11.

152. Yang, M., Baranov, E., Moossa, A. R., Penman, S., and Hoffman, R. M. (2000). Visualizing gene expression by whole-body fluorescence imaging. Proc Natl Acad Sci USA 97, 12278-82.

153. Zhao, W., Chellappa, R., and Krishnaswamy, A. (1998). Discriminant analysis of principal components for face recognition. In Face Recognition: From theory to application, H. e. a. Wechsler, ed. (Amsterdam: ISO (NATO ASI Series)), pp. 73-85.

154. Zuo, J., Niu, Q. W., and Chua, N. H. (2000). An estrogen receptor-based transactivator XVE mediates highly inducible gene expression in transgenic plants. Plant J 24, 265-73.

155. Zuo, J., Niu, Q. W., and Chua, N. H. (2000). Technical advance: An estrogen receptor-based transactivator XVE mediates highly inducible gene expression in transgenic plants. Plant J 24, 265-73.

What is claimed is:

1. A method for detecting gene expression in a sample cell, tissue, organ, or organism, which cell, tissue, organ or organism comprises one or more OCM detectable substances or OCM detectable reporter genes, which method comprises the steps of:
a) acquiring optical coherence microscopy data for said cell, tissue, or organism; and
b) analyzing said acquired data.

2. The method of claim 1 wherein said OCM data comprises signal strength, spatial information, temporal information and voxel information.

3. The method of claim 1 wherein said one or more reporter genes further comprises said one or more reporter genes operatively, linked to one or more regulatory control sequences.

4. The method of claim 1 wherein the one or more gene constructs further comprises one or more selectable marker genes wherein said selectable marker gene(s) are operatively linked to one or more regulatory control sequences.

5. The method of claim 3 wherein said regulatory control sequence is endogenous or exogenous to the cell, tissue, or organism.

6. The method of claim 3 wherein the regulatory control sequence comprises an inducible promoter or a tissue specific promoter.

7. The method of claim 3 wherein the regulatory control sequence is Ca MV 35S promoter.

8. The method of claim 1 wherein step of analyzing the optical coherence microscopy data comprises viewing the optical coherence microscopy data as a 3-dimensional representation.

9. The method of claim 8 wherein said step of viewing further comprises selecting and viewing 2-dimensional portions of the 3-dimensional data.

10. The method of claim 8 wherein said step of viewing further comprises selecting and viewing 3-dimensional subsets of the 3-dimensional data.

11. The method of claim 1 wherein the step of analyzing the optical coherence microscopy data further comprises:
c) selecting binary data comprising spatial voxel information and signal strength corresponding to said spatial voxel information; and
d) performing a histogram analysis on the binary data.

12. The method of claim 11 further comprising the step of selecting and an upper and/or a lower threshold to the signal.

13. The method of claim 11 wherein the step of analyzing the optical coherence microscopy data comprises viewing the optical coherence microscopy image as a 3-dimensional representation of said data.

14. The method of claim 13 wherein said step of viewing further comprises selecting and viewing 3-dimensional subsets of said 3-dimensional representation.

15. The method of claim 13 wherein said step of viewing further comprises selecting and viewing 2-dimensional portions of the 3-dimensional representation.

16. The method of claim 1 wherein said reporter gene comprises phbC.

17. The method of claim 1 wherein said reporter gene comprises phbA, phbB, and phbC.

18. The method of claim 1 wherein said reporter gene comprises phbB and phbC.

19. The method of claim 1 further comprising the steps of:
a. acquiring optical coherence microscopy data for one or more reference cells, tissues or organisms, which reference cells, tissues or organisms do not comprise an OCM detectable reporter gene;
b. comparing said acquired data for said one or more sample cells, tissues, or organisms to said acquired data for said reference.

20. The method of claim 19 wherein said step of comparing further comprises generating a 3-dimensional representation of said acquired data and visually comparing said 3-dimensional representations.

21. The method of claim 20 further comprising selecting and viewing a 2-dimensional portion of said 3-dimensional representation.

22. The method of claim 20 further comprising the step of selecting and viewing 3-dimensional subsets of the 3-dimensional data.

23. The method of claim 19 wherein the step of analyzing the optical coherence microscopy data further comprises:
a) selecting binary data corresponding to the acquired data for each sample and reference, wherein said binary data comprises spatial voxel information and signal strength corresponding to said spatial voxel information; and
b) generating a histogram for each set of binary data wherein said histogram comprises signal unit vs. number of voxels; and
c) comparing the resulting histograms.

24. The method of claim 23 further comprising a mathematical comparison of said histograms.

25. The method of claim 23 further comprising the step of selecting and an upper and/or a lower threshold to the signal.

26. The method of claim 23 wherein the step of analyzing the optical coherence microscopy data comprises viewing the optical coherence microscopy image as a 3-dimensional representation of said data.

27. The method of claim 26 wherein said step of viewing further comprises selecting and viewing 2-dimensional portions of the 3-dimensional representation.

28. The method of claim 26 wherein said step of viewing further comprises selecting and viewing 3-dimensional subsets of said 3-dimensional representation.

29. A method of screening a plurality of cells, tissues, organs or organisms comprising the steps of:
d) acquiring optical coherence microscopy data for one or more reference cells, tissues, organs or organisms
e) generate a reference histogram profile for said reference samples wherein said histogram comprises signal unit vs. average number of voxels;
f) acquire optical coherence microscopy data for one or more sample cells, tissue, organs or organisms;
g) generate a histogram for each sample wherein said histogram comprises signal unit vs. number of voxels;
h) compare each sample histogram to said reference histogram profile.

30. The method of claim 29 wherein said step of comparing further comprises a mathematical comparison between said sample histogram and said reference histogram.

31. The method of claim 30 wherein said reference histogram is subtracted from said sample histogram.

32. The method of claim 29 wherein said reference tissue, cells, organs or organisms comprise wild type or normal tissue, cells, organs or organisms and said samples comprise unknowns, genetically transformed, mutated or otherwise modified cells, tissues, organs or organisms.

33. The method of claim 29 wherein said genetically transformed cells, tissues, organs or organisms comprise one or more OCM detectable reporter gene.

34. The method of claim 33 wherein said one or more reporter genes further comprises said one or more reporter genes operatively linked to one or more regulatory control sequences.

35. The method of claim 34 wherein the one or more of said gene constructs further comprises one or more selectable marker genes wherein said selectable marker gene(s) are operatively linked to one or more regulatory control sequences.

36. The method of claim 34 wherein said regulatory control sequence is endogenous or exogenous to the cell, tissue, or organism.

37. The method of claim 34 wherein the regulatory control sequence comprises an inducible promoter or a tissue specific promoter.

38. The method of claim 34 wherein the regulatory control sequence is Ca MV 35S promoter.

39. The method of claim 33 wherein said reporter gene comprises phbC.

40. The method of claim 33 wherein said reporter gene comprises phbA, phbB, and phbC.

41. The method of claim 33 wherein said reporter gene comprises phbB and phbC.

42. A high throughput OCM system comprising:
a) a tray comprising multiple sample holders arranged in multiple rows; each sample holder designated by a row and column;
b) a multi directional translational device attached to said tray and controlled by a PC;
c) a camera in optical alignment with said tray and connected to said PC;
d) an optical coherence microscopy system in optical alignment with said tray at a known location relative to said camera and also connected to said PC.

43. A method of high throughput OCM screening utilizing the system of claim 1, comprising the steps of:
a) selecting a sample in a sample holder by designating a row and column;
b) centering the sample holder within the within the field of view (FOV) of the camera;
c) acquiring and image from said camera and processing said image data to precisely locate the sample or a desired feature on the sample;
d) calculating the coordinates of the center of the located feature;
e) actuating said sample holder using said translational device to present the feature directly beneath the OCM system,
f) acquire and store said OCM data for said sample.
